(19) United States
(12) Patent Application Publication
(10) Pub. No.: US 2026/0358536 A1
(43) Pub. Date: Nov. 12, 2026

(54) ULTRAVIOLET EMITTING GLASS AND METHOD OF MAKING THEREOF

(71) Applicant: UNIVERSITY OF MASSACHUSETTS, Westborough, MA (US)

(72) Inventors: Mariana LANZARINI-LOPES, Westborough, MA (US); Katrina FITZPATRICK, Westborough, MA (US); Leila Alidokht AKHOONI, Westborough, MA (US)

(21) Appl. No.: 19/489,143

(22) PCT Filed: Jun. 7, 2024
(86) PCT No.: PCT/US2024/033043

§ 371 Date: Dec. 1, 2025

Related U.S. Application Data
(60) Provisional application No. 63/506,985, filed on Jun. 8, 2023.

(51) Int. Cl. G02B 5/02; A61L 2/104

(52) U.S. Cl. CPC G02B 5/0242; A61L 2/104; G02B 5/0268; G02B 5/0278; A61L 2202/11; G02B 2207/101

(57) ABSTRACT

Disclosed herein is an article comprising (a) a light-emitting layer; and (b) one or more UV light sources coupled within the article such that a light beam is side-emitted by the light-emitting layer in a direction substantially perpendicular to a direction of the light beam; wherein the light-emitting layer comprises a plurality of light-scattering elements and exhibits at least 90% transmittance. Also disclosed are methods of making and using the same.

### CROSS-REFERENCE TO THE RELATED APPLICATIONS

This application is an International Patent Application, which claims the benefit of priority to U.S. provisional application No. 63/506,985, filed Jun. 8, 2023, the content of which is incorporated herein in its entirety.

### STATEMENT REGARDING FEDERALLY SPONSORED RESEARCH OR DEVELOPMENT

This invention was made with government support under Grant #N00014-22-1-2540 awarded by the Office of Naval Research, Department of the Navy. The government has certain rights in the invention.

### FIELD

The present patent document is directed generally to devices configured to provide a UV-light emission having disinfecting properties. Methods of making and using the same are also disclosed.

### BACKGROUND

Biofouling on the exterior surface of marine vessels and equipment creates significant operational, functional, and financial hurdles. A biofilm refers to a structured collection of microorganisms that adhere to surfaces through the production of extracellular polymeric substances (EPS). Early-stage biofilm begins to form during initial vessel submersion, developing slime and growing into complex biofouling communities of barnacles, sponges, and tunicates within days. Biological growth and the bio-corrosion associated with biofilms can damage oceanographic equipment and decrease the optical transparency of windows used for cameras and communications devices. Biofilm formation in the United States Navy Fleet costs between 180 and 260 million dollars per year. Increased surface roughness increases hydraulic drag and fuel consumption, while corrosion causes significant irreparable damage to the vessel's components. Moreover, ship biofouling is an important mechanism for the translocation and introduction of non-native species throughout the world, which can impact various economic, ecological, societal, and cultural values.

Current approaches for biofilm prevention on ship hulls involve the application of antifouling biocidal or non-biocidal coatings. Biocidal coatings employ an active agent to kill fouling organisms. Non-biocidal coatings include silicone-based fouling release coatings and those designed to be mechanically resistant to abrasive cleaning. Fouling release coatings reduce the adhesion strength of organisms, resulting in easier removal through hydraulic forces or cleaning. Mechanical cleaning is an effective method to prevent and remove biofilms; however, it is time and cost-intensive and can damage biofilm resistance surfaces while releasing harmful materials into the marine environment.

Germicidal light between 250 and 280 nm wavelengths (UVC) is considered environmentally friendly. This chemical-free disinfection process has been illustrated to successfully prevent marine biofilm formation at extremely low irradiance. UVC light prevents organism replication through damage to the cell's DNA, RNA, and protein structures. In marine ecosystems specifically, surface exposure to UVC radiation resulted in reduced settlement of both coral larvae and barnacles. However, the inability to distribute adequate light across the surface of interest is the main limitation of using UV light for biofilm prevention. Light intensity decays as it moves away from the point source due to diffusion and attenuation. The rapid decrease in the light intensity results in material damage near the source (high irradiance) while simultaneously failing to prevent biofilm within a small distance from the source (low irradiance). Additionally, high turbidity of water diminishes how much UV light reaches the surface.

Accordingly, there is a need in the art for improved methods and devices for the prevention of biofouling that overcome the limitations of prior art methods and devices.

### SUMMARY

In accordance with the purposes of the disclosed materials, compounds, compositions, and methods, as embodied and broadly described herein, the disclosed subject matter, in one aspect, relates to compounds and compositions and methods for preparing and using such compounds and compositions.

In some aspects, disclosed is an article comprising (a) a light-emitting layer; and (b) one or more UV light sources coupled within the article such that a light beam is side-emitted by the light-emitting layer in a direction substantially perpendicular to a direction of the light beam; wherein the light-emitting layer comprises a plurality of light-scattering elements and exhibits at least 90% transmittance.

In other aspects, disclosed is a method of comprising: forming an article by coupling one or more UV light sources such that a light beam is side-emitted by a light-emitting layer in a direction substantially perpendicular to the direction of the light beam; wherein the light-emitting layer comprises a plurality of light-scattering elements and exhibits at least 90% transmittance; wherein the article is at least partially disinfecting and/or anti-biofouling.

Also disclosed is a method comprising: forming any of the disclosed herein articles; exposing the article to a biofouling environment such that the light-emitting layer at least partially faces the biofouling environment, passing a light beam from the one or more UV-light sources to initiate a side-emission of the UV light, and reducing biofouling at at least a portion of a surface of the article that is exposed to the biofouling environment.

Additional advantages will be set forth in part in the description that follows and in part will be obvious from the description or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

The accompanying figures, which are incorporated in and constitute a part of this specification, illustrate several aspects described below.

FIG. 1A is a perspective view of an embodiment of a biofouling-resistant device made in accordance with the present disclosure.

FIG. 1B is a perspective view of a vessel, including an embodiment of a biofouling-resistant device made in accordance with the present disclosure.

FIG. 2 shows a plan view of an exemplary embodiment of an experimental setup of a biofouling-resistant device made in accordance with the present disclosure.

FIG. 3A shows a chart of UV irradiance measurements for slides coated with different concentrations of nanoparticles with a transparent polymer coating, where dilution values of 1 to 0, 1 to 2, 1 to 5, 1 to 10, 1 to 100, and 0 to 1 represent the slides coated with 265, 132.5, 53, 26.5, 2.65, and 0 μg/cm² of nanoparticles, respectively.

FIG. 3B shows a chart of UV irradiance measurements for slides coated with different concentrations of nanoparticles without a transparent polymer coating, where dilution values of 1 to 0, 1 to 2, 1 to 5, 1 to 10, 1 to 100, and 0 to 1 represent the slides coated with 265, 132.5, 53, 26.5, 2.65, and 0 μg/cm² of nanoparticles, respectively.

FIG. 4A shows a chart of transparency of slides coated with different concentrations of nanoparticles without a transparent polymer coating, where dilution values of 1 to 0, 1 to 2, 1 to 5, 1 to 10, 1 to 100, and 0 to 1 represent the slides coated with 265, 132.5, 53, 26.5, 2.65, and 0 μg/cm² of nanoparticles, respectively.

FIG. 4B shows a chart of transparency of slides coated with different concentrations of nanoparticles with a transparent polymer coating, where dilution values of 1 to 0, 1 to 2, 1 to 5, 1 to 10, 1 to 100, and 0 to 1 represent the slides coated with 265, 132.5, 53, 26.5, 2.65, and 0 μg/cm² of nanoparticles, respectively;

FIG. 5 is a chart of the final UV emission profile and the UV spectrum (inset) for the experimental setup used in the submersion shown in FIG. 2. The decrease in irradiance is due to the presence of a sealing polymer.

FIG. 6A shows a photograph of control glasses after immersion for 20 days, showing relative abundance and biofilm coverage thereon.

FIG. 6B shows a photograph of biofouling-resistant devices made in accordance with the present disclosure after immersion for 20 days, showing relative abundance and biofilm coverage thereon.

FIG. 6C shows a graph comparing the average values of the experimental results shown in FIGS. 6A-6B.

FIG. 7A shows a graph of the average number of CFU/cm² of cells of the experimental results.

FIG. 7B shows a graph of the average number of live cells/cm² of the experimental results.

FIG. 7C shows a graph of the average number of dead cells/cm² of the experimental results.

FIG. 8 shows a graph of the concentration of total DNA, unbound, and bound protein in the experimental results.

FIG. 9 shows a schematic of each layer of an exemplary article according to one aspect.

FIGS. 10A-10C show photographs of exemplary articles with and without polymer coatings. FIG. 10A shows an exemplary planar glass substrate coated with a light-emitting layer comprising a plurality of nanoparticles and a layer of a UV-transparent polymer. FIG. 10B shows a lens as a glass substrate coated with a light-emitting layer comprising a plurality of nanoparticles and a layer of a UV-transparent polymer. FIG. 10C shows a control glass lens without coating to show optical transparency.

FIG. 11 shows the UV measurement setup for a 100 mm (diameter)×6 mm (thickness) lens.

FIG. 12 shows an example case design for the submersion of UV side-emitting glass (UEGs) in a marine environment.

FIGS. 13A-13F show the UV emission profiles of UEG at 275 nm.

FIGS. 14A-14C show the UV emission profiles of UEG at 265 nm.

FIGS. 15A-15B show AFM top view images of a glass surface coated with only polymer (a₁) and nanoparticles+polymer (b₁). Mean thickness for the only polymer was 151 nm+/−9 nm and for NP and polymer 422+/−83 nm

FIGS. 16A-16B show corresponding cross-sectional profiles (a₂ and be) of the AFM images shown in FIGS. 15A-15B.

FIGS. 17A-17B demonstrate that silicone sealing decreases UV irradiance from an exemplary article.

FIG. 18 shows that the materials with high absorption coefficients have high refractive indices.

FIGS. 19A-19B depict an exemplary article comprising a PTFE-polymer coating.

FIG. 20 demonstrates that the PTFE coating omitted the adverse effect of silicon on UV irradiance

FIGS. 21A-21C show the experimental setup for the use of an exemplary article.

FIG. 22 shows a significant inhibitory effect (92%) of UEGs on the biofilm growth based on visual assessment.

FIG. 23 shows a decrease in live cell count in biofilm coverage based on CFU.

FIG. 24 shows an exemplary setup of an exemplary article in one aspect of the disclosure.

FIG. 25 shows the UV emission profiles of UEG slides at multiple wavelengths. The slides used as a substrate were 24 mm (W)×110 mm (L)×2 mm (T).

FIG. 26 shows the results of UEGs submerged with Pseudomonas bacteria.

FIG. 27 shows the results of UEGs submerged with mixed marine bacteria.

FIG. 28 shows electrical power per order.

FIG. 29 shows optical coherence tomography (OCT) images of biofilm.

FIG. 30 shows measuring biofilm thickness at the areas covered by biofilm.

FIG. 31 compares the effectiveness of UEG with external UV irradiation.

### DETAILED DESCRIPTION

The materials, compounds, compositions, articles, and methods described herein may be understood more readily by reference to the following detailed description of specific aspects of the disclosed subject matter, and the Examples included therein.

Before the present materials, compounds, compositions, kits, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entirety are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### DEFINITIONS

In this specification and in the claims that follow, reference will be made to a number of terms, which shall be defined to have the following meanings:

As used herein, the terms “optional” or “optionally” mean that the subsequently described event or circumstance can or cannot occur and that the description includes instances where said event or circumstance occurs and instances where it does not.

It is appreciated that certain features of the disclosure, which are, for clarity, described in the context of separate aspects, can also be provided in combination in a single aspect. Conversely, various features of the disclosure, which are, for brevity, described in the context of a single aspect, can also be provided separately or in any suitable subcombination.

As used in the description and the appended claims, the singular forms “a,” “an,” and “the” include plural referents unless the context clearly dictates otherwise.

It is also to be understood that the terminology used herein is for the purpose of describing particular aspects only and is not intended to be limiting. As used in the specification and in the claims, the term “comprising” can include the aspects “consisting of” and “consisting essentially of.” Unless defined otherwise, all technical and scientific terms used herein have the same meaning as commonly understood by one of ordinary skill in the art to which this invention belongs. In this specification and in the claims which follow, reference will be made to a number of terms that shall be defined herein.

For the terms “for example” and “such as” and grammatical equivalences thereof, the phrase “and without limitation” is understood to follow unless explicitly stated otherwise. It is further understood that these phrases are used for explanatory purposes only. It is further understood that the term “exemplary,” as used herein, means “an example of” and is not intended to convey an indication of a preferred or ideal aspect.

The term “or” means “and/or.” Recitation of ranges of values is merely intended to serve as a shorthand method of referring individually to each separate value falling within the range unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context.

All disclosed values also include values that fall within ≤10% variation from the disclosed value unless otherwise indicated or inferred. In other words, if a range of 1 to 10 is disclosed, then a range of about 1 to about 10 is disclosed. In such aspects, it is understood that the amount or value in question can be the exact value or a value that provides equivalent results or effects as recited in the claims or taught herein. That is, amounts, sizes, formulations, parameters, and other quantities and characteristics include both exact values but also approximate, larger or smaller values as desired, reflecting tolerances, conversion factors, rounding, measurement error, and the like, and other factors known to those of skill in the art such that equivalent results or effects are obtained. In some circumstances, the value that provides equivalent results or effects cannot be reasonably determined. In general, an amount, size, formulation, parameter, or other quantity or characteristic is “about,” “approximate,” or “at or about,” whether or not expressly stated to be such. Where “about,” “approximate,” or “at or about” is used before a quantitative value, the parameter also includes the specific quantitative value itself unless expressly stated otherwise.

As used herein, the term or phrase “effective,” “effective amount,” or “conditions effective to” refers to such amount or condition that is capable of performing the function or property for which an effective amount or condition is expressed. As will be pointed out below, the exact amount or particular condition required will vary from one aspect to another, depending on recognized variables such as the materials employed and the processing conditions observed. Thus, it is not always possible to specify an exact “effective amount” or “condition effective to.” However, it should be understood that an appropriate, effective amount will be readily determined by one of ordinary skill in the art.

When a range is expressed, a further aspect includes from the one particular value and to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘x, y, z, or less’ and should be interpreted to include the specific ranges of ‘x,’ ‘y,’ ‘z,’ ‘about x,’ ‘about y,’ and ‘about z’ as well as the ranges of ‘less than x,’ ‘less than y, or ‘less than z,’ or ‘less than about x,’ ‘less than about y, and ‘less than about z.’ Likewise, the phrase ‘x, y, z, or greater’ should be interpreted to include the specific ranges of ‘x,’ ‘y,’ ‘z,’ ‘about x,’ ‘about y,’ and ‘about z’ as well as the ranges of ‘greater than x, greater than y,’ ‘greater than z,’ or ‘greater than about x,’ greater than about y,′ ‘greater than about z.’ In addition, the phrase “‘x’ to ‘y’,” where ‘x’ and ‘y’ are numerical values, also includes “about ‘x’ to about ‘y’.”

| Such a range format is used for convenience and brevity and, thus, should be interpreted flexibly to include not only the numerical values explicitly recited as the limits of the range but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5% but also include individual values (e.g., 1%, 2%, 3%, and 4%) and the sub-ranges (e.g., 0.5% to 1.1%; 0.5% to 2.4%; 0.5% to 3.2%, and 0.5% to 4.4%, and other possible sub-ranges) within the indicated range.

Recitation of ranges of values herein is merely intended to serve as a shorthand method of referring individually to each separate value recited or falling within the range unless otherwise indicated herein, and each separate value is incorporated into the specification as if it were individually recited. Ranges provided herein are understood to be shorthand for all of the values within the range. For example, a range of 1 to 50 is understood to include any number, or combination of numbers, from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or sub-ranges from the group consisting of 10-40, 20-50, 5-35, etc. Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, I-4, and 2-4).

As used herein, the term “composition” is intended to encompass a product comprising the specified ingredients in the specified amounts, as well as any product that results, directly or indirectly, from a combination of the specified ingredients in the specified amounts.

References in the specification and concluding claims to parts by weight of a particular element or component in a composition denote the weight relationship between the element or component and any other elements or components in the composition or article for which a part by weight is expressed. Thus, in a mixture containing 2 parts by weight of component X and 5 parts by weight, components Y, X, and Y are present at a weight ratio of 2:5 and are present in such a ratio regardless of whether additional components are contained in the mixture.

A weight percent (wt. %) of a component, unless specifically stated to the contrary, is based on the total weight of the formulation or composition in which the component is included.

It will be understood that although the terms “first,” “second,” etc., may be used herein to describe various elements, components, regions, layers, and/or sections. These elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of example embodiments.

As used herein, the term “substantially” means that the subsequently described event or circumstance completely occurs or that the subsequently described event or circumstance generally, typically, or approximately occurs.

Still further, the term “substantially” can, in some aspects, refer to at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the stated property, component, composition, or other condition for which substantially is used to characterize or otherwise quantify an amount.

In other aspects, as used herein, the term “substantially free,” when used in the context of a composition or component of a composition that is substantially absent, is intended to refer to an amount that is then about 1% by weight, e.g., less than about 0.5% by weight, less than about 0.1% by weight, less than about 0.05% by weight, or less than about 0.01% by weight of the stated material, based on the total weight of the composition or based on any other calculations as disclosed.

As used herein, the term “substantially,” in, for example, the context “substantially identical” or “substantially similar,” refers to a method or a system, or a component that is at least about 90%, at least about 91%, at least about 92%, at least about 93%, at least about 94%, at least about 95%, at least about 96%, at least about 97%, at least about 98%, at least about 99%, or about 100% by similar to the method, system, or the component it is compared to.

As used herein, the terms “substantially identical reference composition” and “substantially identical reference article” refer to a reference composition or article comprising substantially identical components in the absence of an inventive component. In another exemplary aspect, the term “substantially,” in, for example, the context “substantially identical reference composition” or “substantially identical reference article,” refers to a reference composition or an article comprising substantially identical components and wherein an inventive component is absent or is substituted with a common in the art component.

By “contact” or other forms of the word, such as “contacted” or “contacting,” it is meant to add, combine, or mix two or more compounds, compositions, or materials under appropriate conditions to produce a desired product or effect. The term “react” is sometimes used when “contacting” results in a chemical reaction.

Spatially relative terms, such as,” “beneath,” “below,” “lower,” “above,” “upper,” “upward,” “downward,” “top,” “bottom,” and the like, can be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device can be otherwise oriented (rotated 90 degrees or at other orientations), and the spatially relative descriptors used herein are interpreted accordingly.

Terms such as “proximal,” “distal,” “radially outward,” “radially inward,” “outer,” “inner,” and “side” describe the orientation and/or location of portions of the components or elements within a consistent but arbitrary frame of reference which is made clear by reference to the text and the associated drawings describing the components or elements under discussion. Such terminology can include the words specifically mentioned above, derivatives thereof, and words of similar import.

As used herein, the term “and/or” includes any and all combinations of one or more of the associated listed items. It is also understood that the term “and/or” includes where one or another of the associated listed items is present, and the aspects where both of the associated listed items are present or any combinations of the associated listed items are present.

As used herein, a “UV-transparent material” refers to a material that, in a selected wavelength range, typically has an average percent transmission of least 80%, at least 85%, at least 90%, or at least 95% over the desired wavelength range. For example, a material that is substantially UV-C transparent typically has an average percent transmission of at least 80%, at least 85%, and at least 90% over the UV-C wavelength range.

As described herein, the term “light-emitting layer” refers to a light-side-emitting layer, a light-face-emitting layer, or a combination thereof.

Disclosed herein are materials, compounds, compositions, and components that can be used for, can be used in conjunction with, can be used in preparation for, or are products of the disclosed methods and compositions. These and other materials are disclosed herein, and it is understood that when combinations, subsets, interactions, groups, etc., of these materials, are disclosed while specific reference of each various individual and collective combinations and permutation of these compounds may not be explicitly disclosed, each is specifically contemplated and described herein. For example, if a composition is disclosed and a number of modifications that can be made to a number of components of the composition are discussed, each and every combination and permutation that is possible are specifically contemplated unless specifically indicated to the contrary. Thus, if a class of components A, B, and C are disclosed and a class of components D, E, and F and an example of a combination composition A-D are disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from the disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure, including, but not limited to, steps in methods of making and using the disclosed compositions. Thus, if there are a variety of additional steps that can be performed, it is understood that each of these additional steps can be performed with any specific aspect or combination of aspects of the disclosed methods, and that each such combination is specifically contemplated and should be considered disclosed.

While aspects of the present invention can be described and claimed in a particular statutory class, such as the system statutory class, this is for convenience only, and one of ordinary skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to the arrangement of steps or operational flow, plain meaning derived from grammatical organization or punctuation, or the number or type of aspects described in the specification.

The present invention may be understood more readily by reference to the following detailed description of various aspects of the invention and the examples included therein and to the Figures and their previous and following description.

### Articles

In certain aspects, provided is an article comprising (a) a light-emitting layer; and (b) one or more UV light sources coupled within the article such that a light beam is side-emitted by the light-emitting layer in a direction substantially perpendicular to a direction of the light beam; wherein the light-emitting layer comprises a plurality of light-scattering elements and exhibits at least 90% transmittance. In yet still further aspects, the light-emitting layer can exhibit at least 91% transmittance, at least 92% transmittance, at least 93% transmittance, at least 94% transmittance, at least 95% transmittance, at least 96% transmittance, at least 97% transmittance, at least 98% transmittance, at least 99% transmittance, at least 99.5% transmittance, or at least 99.9% transmittance. In still further aspects, the light-emitting layer can exhibit transmittance in a range of 90% to 100%, including 90%-99%, 90%-98%, 90%-98%, 90%-97%, 90%-96%, 90%-95%, 90%-95%, 90%-93%, 90%-92%, 91%-100%, 92%-100%, 93%-100%, 94%-100%, 95%-100%, 96%-100%, 97%-100%, 98%-100%, and so on.

In certain aspects, the light-emitting layer can be used as a separate layer. In yet other aspects, the light-emitting layer can be positioned on a substrate. In certain aspects, the substrate can be transparent or non-transparent. In yet still further aspects, the light-emitting layer can be at least partially embedded within the substrate. For example, and without limitations, when the substrate is transparent, the light-emitting layer can be at least partially embedded within the bulk of the substrate adjacent to at least one surface of the substrate. In such exemplary and unlimiting aspects, the light-emitting layer can be formed during the formation of the substrate itself, or it can be embedded into a formed substrate by any method known in the art.

In certain aspects, the plurality of light-scattering elements comprise a plurality of particles. In yet still further aspects, the plurality of particles is a plurality of nanoparticles. In certain aspects, the plurality of nanoparticles can have any size to achieve the desired light emission. In some exemplary and unlimiting aspects, the nanoparticles can have a size of 50 nm to 500 nm, including exemplary values of 80 nm, 100 nm, 120 nm, 150 nm, 180 nm, 200 nm, 220 nm, 250 nm, 280 nm, 300 nm, 320 nm, 350 nm, 380 nm, 400 nm, 420 nm, 450 nm, and 480 nm. It is understood that the plurality of nanoparticles can have any size that falls between any two foregoing values or falls within a range formed by any two foregoing values. For example, and without limitations, the size of the nanoparticles can be 50 nm to 450 nm, 50 nm to 400 nm, 50 nm to 350 nm, 50 nm to 300 nm, 50 nm to 250 nm, 50 nm to 200 nm, 50 nm to 150 nm, 50 nm to 100 nm, 100 nm to 500 nm, 150 nm to 500 nm, 200 nm to 500 nm, 250 nm to 500 nm, 300 nm to 500 nm, 350 nm to 500 nm, 400 nm to 500 nm, or 450 nm to 500 nm and so on. In still further aspects, the size of the light-scattering elements can be chosen at least in part upon the light wavelength being employed to achieve the desired scattering of the UV light. In certain aspects, the desired size of the light-scattering elements is chosen to provide substantially equal and uniform scattering at all wavelengths in the desired range (which is disclosed below). Yet, in other aspects, the size of the light-scattering elements can be chosen such that the scattering is not uniform at all the wavelengths.

It is understood that any nanoparticles that can provide the desired effect can be used. In certain aspects, the nanoparticles can comprise silica. Yet in other aspects, the plurality of nanoparticles can comprise glass, metal oxides, zeolites, polymer particles (for example, PMMA, PVC, acrylates, and so on), or any combination thereof. In certain aspects, the nanoparticles can be functionalized or unfunctionalized. In certain aspects, silica nanoparticles can be functionalized with amine groups forming aminated silica nanoparticles. In such exemplary and unlimiting aspects, the aminated silica nanoparticles can exhibit a positive charge. In such aspects, when the nanoparticles have at least a partial charge, the nanoparticles can be disposed on any substrate using electrostatic deposition. In still further aspects, instead of silica nanoparticles, silica beads of any of the disclosed sizes can be used as a light-scattering element.

In still further aspects, when the light-emitting layer is at least partially embedded into the transparent substrate, the plurality of light-scattering elements can comprise one or more of a plurality of particles (or nanoparticles), defects, air bubbles, or a combination thereof. Any of the disclosed above plurality nanoparticles can be used herein. It is understood that other defects and air bubbles can also be formed during glass (when used as a transparent substrate, for example) manufacturing.

In certain aspects, the light-emitting layer can have a thickness of 50 nm to 5 cm, including exemplary values of 100 nm, 250 nm, 500 nm, 750 nm, 1 micron, 25 microns, 50 microns, 100 microns, 250 microns, 500 microns, 750 microns, 1 mm, 5 mm, 1 cm, 1.5 cm, 2 cm, 2.5 cm, 3 cm, 3.5 cm, 4 cm, 4.5 cm, or 4.9 cm. It is understood that the thickness of the layer can have any value that falls between any two foregoing values or within a range that is formed by any two foregoing values. For example, and without limitations, the thickness of the light-emitting layer can be 50 nm to 2 cm, 50 nm to 1 cm, 50 nm to 900 microns, 50 nm to 500 microns, 50 nm to 200 microns, 50 nm to 50 microns, 50 nm to 900 nm, 50 nm to 500 nm, 50 nm to 100 nm, 100 nm to 500 nm, 200 nm to 200 microns, 200 nm to 5 cm, 300 nm to 5 cm, 500 nm to 5 cm, 1 micron to 5 cm, 50 microns to 5 cm, 500 microns to 5 cm, 1 cm to 5 cm and so on.

In still further aspects, if the light-emitting layer is disposed on the substrate, the substrate (either transparent or non-transparent) can have any thickness that is needed for the desired application. In certain and unlimiting aspects, the substrate can have a thickness of 250 microns to 50 cm, including exemplary values of 500 microns, 750 microns, 1 mm, 1 cm, 5 cm, 10 cm, 20 cm, 30 cm, 40 cm, and 45 cm. It is understood that the thickness of the substrate (transparent or non-transparent) can have any value that falls between any two foregoing values or within a range that is formed by any two foregoing values. For example, and without limitations, the thickness of the substrate can be 250 microns to 45 cm, 250 microns to 40 cm, 250 microns to 30 cm, 250 microns to 20 cm, 250 microns to 10 cm, 250 microns to 1 cm, 250 microns to 750 microns, 300 microns to 50 cm, 500 microns to 50 cm, 750 microns to 50 cm, 1 cm to 50 cm, 25 cm to 50 cm, and so on.

In certain aspects, the one or more UV sources can be optically coupled with the light-emitting layer. Yet, in other aspects, the one or more UV light sources are optically coupled with the transparent substrate. In yet still further aspects, the one or more UV light sources can be optically coupled with a transparent substrate and the light-emitting layer.

In certain aspects, if the transparent substrate is present it can behave as an optical waveguide such that the light beam propagates along a longitudinal axis of the transparent substrate. Again, it is understood that the thickness of the transparent substrate can have any of the disclosed above values.

In still further aspects, the light-emitting layer can have any irregular or regular shape and geometry. In still further aspects, the substrate, if present, can also have any regular shape and geometry. In still further aspects, the article itself can have a regular or irregular form. In still further aspects, at least a portion of the light-emitting layer substantially conforms to at least one surface of the substrate. In still further aspects, the shape of the layer and/or substrate and/or final article can be planar, curved, concaved, convex, spherical, hemispherical, ellipsoid, having one or more edges, being substantially absent of sharp edges, or can comprise sharp edges, 2 dimensional or 3 dimensional, or any combination thereof. It is further understood that the layer does not have to be fully continuous. For example, the light-emitting layer can be made as a mesh or weave or a grid-like pattern. In yet still further aspects, the light-emitting layer can be disposed on a specific portion of the substrate while keeping other portions free of the light-emitting layer, thus creating a desired pattern for the desired light emission. In still further aspects, the substrate is not an optical fiber. In yet other aspects, the light-emitting layer is only disposed on one surface of the substrate. In yet still further aspects, the light-emitting layer is disposed on one or more surfaces of the substrate. In still further aspects, the light-emitting layer does not form a shell around a substrate core.

In still further aspects, the one or more UV sources can operate at any wavelength in a range of 200 nm to 400 nm, including exemplary values of 210 nm, 215 nm, 220 nm, 225 nm, 230 nm, 235 nm, 240 nm, 245 nm, 250 nm, 255 nm, 260 nm, 265 nm, 275 nm, 280 nm, 285 nm, 290 nm, 295 nm, 300 nm, 305 nm, 310 nm, 315 nm, 320 nm, 325 nm, 330 nm, 335 nm, 340 nm, 345 nm, 350 nm, 355 nm, 360 nm, 365 nm, 375 nm, 380 nm, 385 nm, 390 nm, and 395 nm. In still further aspects, the wavelength can be any wavelength that falls between any two foregoing values or in a range that is formed by any two of the foregoing values. For example, and without limitations, the operating wavelength can be 200 nm to 380 nm, 200 nm to 350 nm, 200 nm to 300 nm, 220 nm to 380 nm, 250 nm to 300 nm, 250 nm to 280 nm, 265 nm to 300 nm, and so on.

In still further aspects, if more than one UV source is present, each of the UV sources can be operated independently on the same or different wavelength. In certain aspects, if more than one UV source is present all UV sources operate on the same wavelength. Yet, in other aspects, if more than one UV source is present, some of the UV sources operate on the same wavelength, while others operate at a different wavelength. Yet in still further aspects, if more than one UV source is present all UV sources operate on different wavelengths. In still further aspects, the one or more UV sources can be positioned anywhere such that the desired coupling effect is achieved. In still further aspects, the one or more UV sources can be controlled by a control unit. In such exemplary and unlimiting aspects, the control unit can adjust the operating wavelengths of the one or more UV sources over the operating time as desired. For example, at the beginning of the use of the article, one or more UV sources can operate a first predetermined wavelength that can be then changed to a second predetermined wavelength during the article operation if desired. The operating wavelength can be changed and adjusted as needed.

In still further aspects it is understood that any known UV sources can be used. For example, the UV source can be xenon lamp, mercury lamp, laser, light-emitting diode (LED), or any combination thereof. In still further aspects, the UV source is a UV-C source. In yet still further aspects, the UV source is an LED. In still further aspects, the UV source can be optically coupled to a heat sink if needed.

In still further aspects, the light-emitting layer comprises one or more polymer layers. In certain aspects, the plurality of light-scattering elements can be embedded within at least one polymer layer. In still further aspects, the one or more polymer layers are at least substantially UV-transparent. In yet still further aspects, the one or more polymer layers can exhibit a minimal absorption of the UV light in the desired range of the wavelengths. In certain aspects, the one or more polymers are transparent polymers. In still further aspects, the one or more polymers can comprise CYTOP™, any transparent fluoropolymers, PMMA, PVC, acrylates, silicones, or any combination thereof. It is further understood that if more than one polymer layer is present, these polymers can be positioned in any order as long as the light-emitting layer remains substantially transparent (at least 90% transmittance) in the desired range of the UV wavelengths.

In still further aspects, the one or more polymer layers of the light-emitting layer can also comprise a sealing polymer. In such aspects, this polymer can be disposed at locations that could be exposed to a delaminating environment or an environment that can negatively affect the article structure. For example, in aspects where the article is used in marine or any other aquatic environment, the sealing layer can be used to prevent delamination of the light-emitting layer (especially if it is disposed on the substrate). In other aspects, the sealing layer can also be used to seal UV sources themselves or coupling routes of the UV sources with the light-emitting layer from exposure to the outside environment. In yet other aspects, the sealing layer can also be used to seal any additional electronic components, if present, in the article from the outside environment.

In still further aspects, the one or more polymer layers are configured to be a host material for the plurality of the light-scattering elements that can be disposed within the polymer in any concentration or pattern. In such exemplary and unlimiting aspects, the nanoparticle can be disposed within the pre-polymer solution, which can then be polymerized together with the nanoparticles. Yet, in other aspects, other known methods can be used to incorporate the nanoparticles within the polymer.

In still further aspects, when the transparent polymer is used as a host, an additional transparent polymer layer can be disposed onto. Yet in still further aspects, if the transparent polymer is used as a host for the plurality of light-scattering elements no additional polymer layers are disposed onto it. It is understood, however, that the host polymer material is UV transparent and can be selected from any disclosed above polymers. In still further aspects, the nanoparticles can be first disposed on the surface, and the transparent polymer layer can be disposed onto to form the light-emitting layer. In certain aspects, especially when the sealing polymer is used in a specific location, the light-emitting layer can further comprise at least one of the one or more of the polymer layers that is configured to substantially prevent UV light absorption. For example, an additional layer of PTFE (Teflon®) can be present. Surprisingly, it was found that a layer of polytetrafluoroethylene (PTFE) can cure the negative effects of the silicon coating (or sealing layer) (such as a decrease in UV irradiance from the article). It was also found that the PTFE layer can prevent side emissions in areas that are not desired.

It is understood that the positioning and composition of the one or more polymer layers can be selected based on the desired application of the article and the optical properties of each polymer layer.

In still further aspects, the light-scattering elements can be present in the light-emitting layer at any concentration as desired. In certain aspects, the light-scattering elements are dispersed uniformly with the layer along any axis of the layer (length, width, and/or thickness of the light-emitting layer). In still further aspects, the light-scattering elements can form a concentration gradient along the thickness of the layer and/or length of the layer and/or width of the layer. In still further aspects, the light-emitting layer can comprise a localized dispersion of the plurality of light-scattering elements as desired.

In still further aspects, the plurality of light-scattering elements is present in an amount of greater than 0 wt % to less than 100 wt % of the light-emitting layer. For example, the plurality of light-scattering elements can be present in an amount of at least 0.01 wt %, at least 0.05 wt %, at least 0.1 wt %, at least 0.5 wt %, at least 1 wt %, at least 1.5 wt %, at least 2 wt %, at least 5 wt %, at least 10 wt %, at least 20 wt %, at least 30 wt %, at least 40 wt %, at least 50 wt %, or at least 60 wt % of the light-emitting layer.

In some aspects, the concentration of the plurality of light-scattering elements can be about 2.65 g/cm² to about 265 μg/cm², including exemplary values of 5 μg/cm², 10 μg/cm², 15 μg/cm², 20 μg/cm², 25 μg/cm², 30 μg/cm², 40 μg/cm², 50 μg/cm², 60 μg/cm², 70 μg/cm², 80 μg/cm², 90 μg/cm², 100 μg/cm², 125 μg/cm², 150 μg/cm², 175 μg/cm², 200 g/cm², 225 μg/cm², 250 μg/cm², and 260 μg/cm². In still further aspects, the concentration of the plurality of light-scattering elements can fall between any two foregoing values or in a range formed by any two foregoing values. For example, and without limitations, the concentration of the plurality of light-scattering elements can be 5 μg/cm² to about 265 μg/cm², 20 μg/cm² to about 265 μg/cm², 50 μg/cm² to about 265 μg/cm², 100 g/cm² to about 265 μg/cm², 150 μg/cm² to about 265 μg/cm², 2.65 μg/cm² to about 250 μg/cm², 2.65 μg/cm² to about 200 μg/cm², 2.65 μg/cm² to about 150 μg/cm², 2.65 μg/cm² to about 100 μg/cm², 2.65 μg/cm² to about 80 μg/cm², and so on. In yet other aspects, the silica nanoparticles can have a concentration of about 26.5 μg/cm².

In still further aspects, the disclosed article can be a ship hull, lenses, windows, sensors, surgical table, optical equipment, heat exchanger, medical equipment, aerospace equipment, bathroom equipment, oceanographical equipment, appliances, wound healing article, or a combination thereof. In still further aspects, the disclosed articles can be used in camera lenses, optical equipment, sensors, surgical tables, clean rooms, dental surfaces, medical surfaces, aerospace surfaces, bathroom surfaces, oceanographical surfaces, agricultural equipment, water treatment surfaces, in heat exchangers, in various appliances and equipment, and so on.

In still further aspects, any of the disclosed herein articles are disinfecting. It is understood that, in some aspects, the articles are self-disinfecting. Yet, in other aspects, the articles can disinfect other objects, surfaces, and items if needed. Yet, in other aspects, the articles can disinfect other objects, surfaces, and items that are in close vicinity to the articles disclosed herein. In still further aspects, when the article is a wound healing article, the article can disinfect and treat a wound that it is applied to. It is understood that the articles disclosed herein can be or can be used anywhere wherein microbial organisms can attach and grow. Such articles can disturb or prevent the growth of microbial organisms, thereby disinfecting the desired surfaces. The article can also be used in curing and advanced oxidation processes.

In still further aspects, when the article is exposed to a biofouling environment such that at least a portion of the light-emitting layer faces the biofouling environment, the article exhibits reduced biofouling as compared to a substantially identical reference article in the absence of the light-emitting layer. In certain aspects, the article itself is anti-biofouling.

In certain aspects, the articles disclosed herein can be used in marine environments to prevent the growth of biofilms on marine vehicles or oceanographic equipment.

Also disclosed are disinfectant systems comprising any of the articles disclosed herein.

### Methods

Also disclosed herein are methods of making the described above articles. In such aspects, the methods comprise forming an article by coupling one or more UV light sources such that a light beam is side-emitted by a light-emitting layer in a direction substantially perpendicular to the direction of the light beam; wherein the light-emitting layer comprises a plurality of light-scattering elements and exhibits at least 90% transmittance; wherein the article is at least partially disinfecting and/or anti-biofouling.

As disclosed above, the light-emitting layer can be disposed on a transparent or non-transparent substrate. In yet other aspects, the light-emitting layer can be at least partially embedded in the transparent layer.

In still further aspects, the light-emitting layer can comprise one or more polymer layers. In certain aspects, the plurality of light-scattering elements at least partially are embedded in one or more polymer layers. It is understood that the plurality of light-scattering elements can be dispersed in the light-emitting layer as desired. In certain aspects, the plurality of light-scattering elements is uniformly dispersed in the light-emitting layer. In other aspects, the light-emitting layer comprises a concentration gradient of the plurality of light-scattering elements along the length, width, and/or depths of the light-emitting layer. In still further aspects, the light-emitting layer comprises a localized dispersion of the plurality of light-scattering elements.

Any of the disclosed above light-scattering elements can be used to form the disclosed herein articles. The light-scattering elements disclosed herein can be of any size as disclosed above. In still further aspects the light-scattering elements can be dissolved in a polymer solution that can be polymerized to form the desired layer. In still further aspects, the light-scattering elements can be electrostatically deposited on any surface as disclosed in the examples below. In still further aspects, the light-scattering elements embedded in the polymerized polymer to form the light-emitting layer. In still further aspects, the light-emitting layer itself can also be formed on a substrate spraying, deposition from a solution, spin coating, doctor blade coating, dip coating, 3D printing or any combination thereof.

In still further aspects, the one or more polymers present in the article can be disposed by any known in the art methods. It is understood that the light-emitting layer can be formed such that it is embedded in any of the disclosed herein polymer layers, or any of the disclosed herein polymer layers can be disposed on the nanoparticles as described above. In still further aspects, the sealing layer can be applied in the desired locations by any known in the art methods. In yet still further aspects, if desired a layer PTFE can also be applied to negate undesirable effects of the sealing layer.

Any of the disclosed herein UV sources can be used to form the desired article. Any of the disclosed above couplings of the one or more UV sources in the article can be utilized.

Also disclosed herein are methods comprising; forming any of the disclosed herein articles; exposing the article to a biofouling environment such that the light-emitting layer at least partially faces the biofouling environment, passing a light beam from the one or more UV-light sources to initiate side-emission of the UV light, and reducing biofouling at at least a portion of a surface of the article that is exposed to the biofouling environment. In still further aspects, the article is self-disinfecting. In yet other aspects, the article is anti-biofouling.

Also disclosed is a method comprising: exposing any of the disclosed herein articles to a biofouling environment such that the light-emitting layer at least partially faces the biofouling environment, passing a light beam from the one or more UV-light sources to initiate a side emission of the UV light, and reducing biofouling at at least a portion of a surface of the article that is exposed to the biofouling environment

### EXAMPLES

The following examples are set forth below to illustrate the methods and results according to the disclosed subject matter. These examples are not intended to be inclusive of all aspects of the subject matter disclosed herein but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, the temperature is in ° °C or is at ambient temperature, and pressure is at or near atmospheric. There are numerous variations and combinations of reaction conditions, e.g., component concentrations, temperatures, pressures, and other reaction ranges and conditions, which can be used to optimize the product purity and yield obtained from the described process. Only reasonable and routine experimentation will be required to optimize such process conditions.

### Example 1

In this example, disclosed is an approach where UV light has glowed from the inside of windows to prevent biological attachment and growth. Glass substrates such as windows can be modified to externally emit UV light and eliminate biological growth on their surface. In this example, nanoparticle-enabled waveguides are formed. UVC light (265 nm), launched from an LED, travels through the glass core and is scattered by the nanoparticles on the surface, resulting in side-emission into its surrounding environment (water/air). When total internal reflection (TIR) occurs within a waveguide, an evanescent wave, or electromagnetic disturbance, is generated through the sides of the substrate. Adding scattering nanoparticles onto the glass surface disturbs the electromagnetic wave, causing the light to leave (leak out) the waveguide substrate, resulting in side-emission and glowing effects.

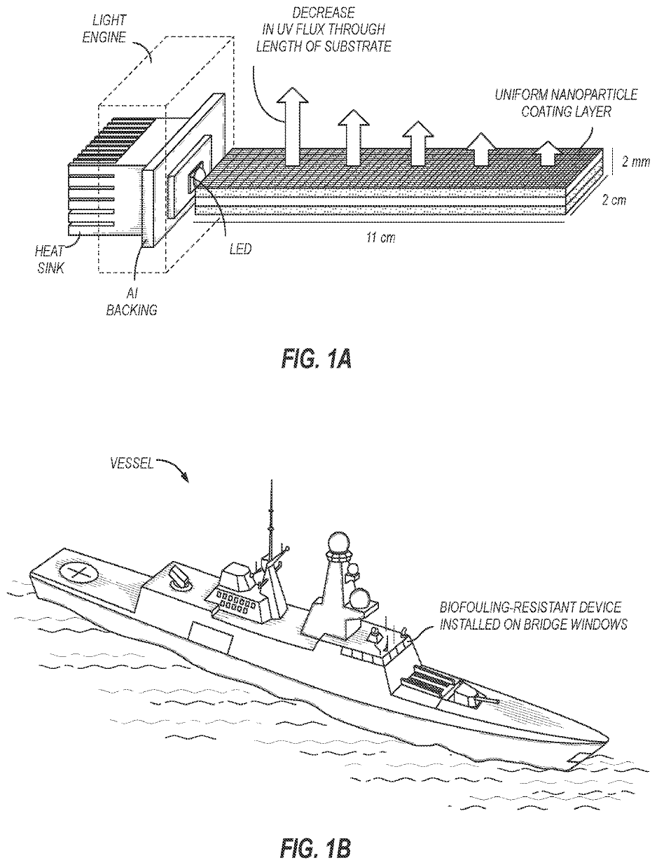

Previously, side-emitting optical fibers (SEOFs) were fabricated by modifying a traditional optical fiber with highly scattering particles (silica spheres>200 nm in diameter) and attaching a UV 265 nm LED to the fiber. SEOFs can effectively inactivate planktonic bacteria (2.9 log inactivation of Escherichia coli at a delivery dose of 15 mJ/cm²) and prevent bacteria growth on surfaces (inhibition zone of 3 cm at a delivery dose of ~4.3 mJ/cm²). However, these studies were only limited to cylindrical optical fibers. The examples disclosed herein show that the side-emitting layers can have any desired geometry or can be formed on any surface having the desired geometry without any limitations. In this example, the UVC emitting glass (UEG) was fabricated to emit UV light throughout its length (FIG. 1A). The UEG was deployed in a marine environment for 20 days to assess the ability of UEGs to prevent biofilm when compared to a similar non-UEG control.

Besides windows and portholes on marine vessels, the biofouling-resistant device and methods may be incorporated into any transparent window. For example, in optical equipment for cameras, signal transmission (UV through IR) and detection equipment, as well as window surfaces. Additionally, through slight modifications to this technology (towards thin film), the device and method disclosed herein may be adapted for biofilm prevention on any surface, both in marine and other ecosystems/environments.

In this example, and referring to FIG. 1A, a biofouling-resistant device made in accordance with the disclosure herein, is shown generally. The biofouling-resistant device comprises a transparent media, such as glass, having an outer surface with a light dispersive coating deposited thereon. The light dispersive coating may include a layer of silica nanoparticles ranging in size from at least 50 nm to at least 100 nm to at least 200 nm in diameter. The silica nanoparticles may be spherical. A UV light source is optically connected to the transparent media. In some aspects, an LED can be optically connected to the transparent media. The light source may further include thermal management solutions, such as an Al backing plate and/or heat sink connected to the LED. Other thermal management techniques, as known in the art, may be used. The dispersive coating disperses UV light emitted from the UV light source onto the outer surface of the transparent media, thereby reducing biofouling. In some aspects, the UV light source can emit light in wavelengths between about 250 nm and about 280 nm, and in other aspects, at about 265 nm. In some aspects, the concentration of silica nanoparticles may be from about 2.65 μg/cm² to about 265 g/cm². In yet other aspects, the silica nanoparticles can have a concentration of about 26.5 μg/cm². An optional silicone binder coating may overlay the light-dispersive coating to protect the light-dispersive coating.

The biofouling-resistant devices may be made by providing a transparent media having an outer surface. The outer surface may be coated through a technique such as electrostatic deposition with a light dispersive coating, such as aminated silica nanoparticles. The UV light source is optically connected to the transparent media, either before or after the coating step. In some aspects, the LED can be optically connected to an edge of the transparent media.

Referring to FIG. 1B, the biofouling-resistant device may be incorporated into a marine environment that is highly prone to biofouling, such as a window on the bridge of a marine vessel. Besides windows and portholes on marine vessels, the biofouling-resistant device and methods may be incorporated into any transparent window. For example, in optical equipment for cameras, signal transmission (UV through IR) and detection equipment, as well as window surfaces. Additionally, through slight modifications to this technology (towards thin film), the device and method disclosed herein may be adapted for biofilm prevention on any surface, both in marine and other ecosystems/environments.

### Example 2: Experimental Methods

Fabrication of UV-emitting glass (“UEG”): UV transparent quartz slides measuring 24×110 mm and 2 mm thick were modified to emit UV light through a three-step method. Step 1 involved cleaning the slides at room temperature by immersing them in acetone (99.5%) and sonicating the solution for 10 min to remove any deposits from the exposed surfaces. Step 2 included the application of scattering nanoparticles onto the cleaned slides by electrostatic deposition mechanism. Aminated silica spheres suspended in ethanol with a diameter of 200 nm (nanoComposix, San Diego, CA, 10 mg/mL, SIAN200) were selected due to the low absorptivity and high scattering coefficient at UV 265 nm. Positively charged aminated spheres facilitated the attachment to the negatively charged glass slide surface. The nanoparticle suspension was sonicated for 10 minutes before application. Approximately 700 μL of the suspension was gently pipetted onto the slide to uniformly cover the 26.5 cm² surface area of the slide before drying for 2 hours. UEGs with different mass coverage (μg/cm²) of nanoparticles on the slides were fabricated by diluting the nanoparticle suspension with Ethyl Alcohol 100% (Decon Laboratories, Inc. PA). In Step 3, the slide was coated with a UV transparent polymer by semi-print screen technique. Following sonication for 10 minutes, 300±20 μL polymer (for example, Cyclic Transparent Optical Polymer (CYTOP™) (AGC Chemicals, USA)) was dragged along the exposed facet of the glass slide and dried at room temperature overnight.

A minimum coating thickness was desired while ensuring complete coverage of the substrate and nanoparticles. CYTOP is a fluoropolymer characterized by its amorphous structure, allowing for remarkable transparency (>95% for 1 mm) over a wide spectral range (200-2000 nm), and is well-suited for functioning as an optical thin film coating. The thickness of the coating material was measured by AFM profilometry using a Jupiter XR Atomic Force Microscopy (AFM) (Oxford Asylum Research, UK) across a scratch made by a brass pin on the coating layer. When scanning, measurements of the material derived from scratching were avoided by selecting areas free of debris when possible. The Instrument was operating in tapping mode using Super-Sharp PointProbe-Plus®-NCHR Silicon tips (NANOSENSORS™, Switzerland). These measurements were repeated four times at different locations, and the average value was reported.

Establishing optical transparency vs. UV emission profile: A series of 12 slides was prepared to investigate the optical transparency and UV emission profile resulting from changes in nanoparticle concentration. The first six slides were fabricated with varying concentrations of nanoparticles, including 265, 132.5, 53, 26.5, 2.65, 0 μg/cm², prepared from 1:0, 1:2, 1:5, 1:10, and 1:100, 0:1 dilutions of nanoparticle suspension, respectively. Each of these slides was then coated with a polymer layer. The remaining six slides were coated with nanoparticles at the same mass coverage without the polymer coating. Optical transparency and UV emission profiles were subsequently characterized for the fabricated UEGs.

The optical transparency of the slides was measured by the U.S. Naval Research Laboratory (NRL) by positioning a collimated light source in front of the substrate followed by a detector behind it. Measurements were carried out in the Vis to NIR spectra (400-2500 nm) using Flame and NIR Quest spectrometers (Ocean Insight, USA) working in tandem. The light sources used in the measurements were provided by Ocean Insight light sources (DH-2000-FHS-DUV-TTL for Vis and HL-2000-HP-FHSA for NIR, respectively), directed onto the slide using a sample 74-ACH adjustable collimating lens holder, modified with custom-made 3D printed panel brace and 74-UV collimating lenses. The intensity of light passing through the slides was collected and recorded using the Ocean View software package, and the % Transmission was calculated during data processing using the MATLAB software package using control (uncoated) slides for background signals. Visual Resolving Power evaluations were conducted using a USAF 1951 Optical Calibration Target printed at 8.5″×11″ scale. Comparisons are made to appropriate uncoated blank.

The UVC emission profile measurements were conducted using a spectrophotoradiometer (AvaSpec-2048L, Avantes, Louisville, CO USA), calibrated over the wavelength range of 200 to 1100 nm. UEGs were placed in a slide holder case (section 2.3.1) in a perpendicular position to the UV LED lamp. The UV irradiance (ρW/cm²) was measured along the length and perpendicular distance from the UEG surface by placing the spectrophotoradiometer's sensor tip (5 mm²) normal to the surface at a 1 mm distance. Intensity was measured at distances of 1, 3, 5, 7, and 9 cm from the UV lamp (for example, LED). The total irradiance was obtained by integrating the output spectrum between 240 nm and 300 nm. Triplicate measurements were taken at each position along independent UEGs. The output intensity was corrected for the dark data for correct full-width half maximum (FWHM) calculations. The reflectance of light by the benchtop was measured by inverting the spectroradiometer tip away from the light source and normal to the benchtop surface. No signal was measured, indicating that the surrounding material had no influence on the light distribution profile.

The absorbance of air and light reflectivity of the benchtop were negligible and should not influence the light distribution profile. However, the light attenuation profile would change when the UEGs are placed in water.

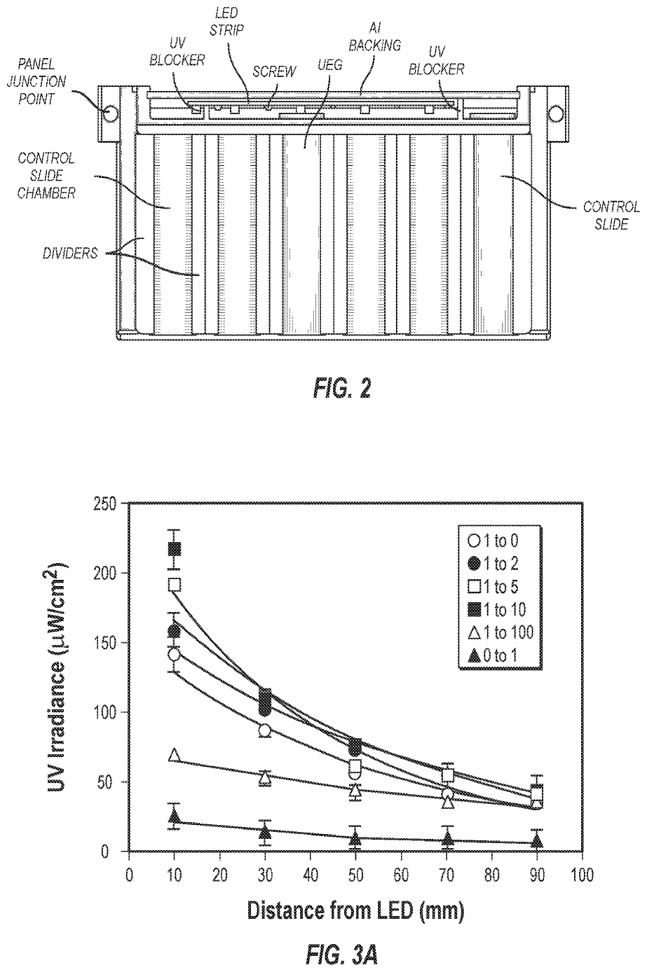

Submersion-Case design: The submersion cases were 3D-printed by UMass Advanced Digital Design and Fabrication (ADDFab) 3D printing facilities. The material used for the case printing was nylon-12 (PA2200) with a density (laser sintered) of 930 kg/m³ and certified for biological tests (USP class: VI). Each case contained six chambers housing two control slides and 4 UEG (nanoparticles concentration: 26.5 μg/cm²) (FIG. 2).

The controls included polymer-only slides with no UV. Each chamber dimension was approximately 25 mm×3 mm×100 mm. 3D-printed side brackets secured the water irradiance against one side of the chamber so that only one side of the slides was exposed to the marine environment. Non-exposing sides of all slides were coated with polymer.

Coupling UV LED to UEG: The 3D-printed cases contained specific areas designed to accommodate the LED strip, aluminum backing, heat sink, and electrical connections and wiring (FIG. 2). Strips of four UV LEDs with 30-degree lenses and emission peak of 265 nm were purchased from Violumas, San Diego, CA. The maximum forward current and forward voltage were 700 mA and 24 V, respectively. The LED strip was screwed to the aluminum backing and placed in the light housing space. A butt-coupling approach was used between LED and UEGs. The UEGs were secured inside the light engine by a silicone binder.

The LED set up produces up to 2-4 J/s of heat during the submersion. During submersion, the marine environment was used as the heat sink. However, during UV profile measurements, four 22 mm×22 mm×7 mm anodized aluminum heatsinks (Easycargo, Amazon, Seattle, WA, USA) were attached to the Al backing to ensure proper semiconductor cooling. A fan (HT 900, Honeywell, Charlotte, NC, USA) was directed to the LED to further dissipate heat. A 700 mA current was delivered to the UV LEDs for all experiments. The output wattage of each LED was 71 mW. During submersion, the light engine was sealed with a silicone binder to avoid water infiltration. The cases were connected to an external power source, and all electronics except the LEDs were kept above water. Cases were mounted to a submersion platform via panel junction points and submerged for 20 days.

### Example 3: Biofilm Formation Analysis

Visual assessment of biofilm formation: The American Society for Testing and Materials (ASTM D6990) standard was used to qualitatively assess biofouling coverage when UEGs and control slides were subjected to immersion conditions in a marine environment. The assessment was performed in the field immediately after slide retrieval to estimate the total coverage of biofilm. Visual assessment included observation of color differences that may signify different types of organisms and the softness of coverage (a single colony of coverage vs. multiple colonies of coverage). Each slide was photographed and divided into a grid arrangement to define observation areas. Prior to retrieval, submersed cases were gently agitated to remove any silt or slime from the slides surface.

Pour plate method (viable cell quantification): After retrieval, each slide was divided into 3 equal parts, namely p₁, p₂, and p₃. Among these parts, p₁ was closest to the UV LED. Biofilms were sterilely extracted from the slide surface using a sterile cell scraper and by reproducible brushing pattern. The extracted biofilm was suspended in a 10 mL sterile phosphate buffer solution (PBS) to prevent osmotic shock and dispersed via combined action of vortex-ultrasound-vortex (each for 1 min). The resulting suspension was designated as BS (biofilm suspension). PBS was prepared by dissolving one BupH™ PBS Pack (Fisher Scientific, USA) in a final volume of 500 mL deionized water. The resulting suspension was designated as BS (biofilm suspension).

To prepare Marine Agar, 37.4 g of Marine Broth powder (Sigma-Aldrich, 76448) and 10 g of Agar (Sigma-Aldrich, A1296) were dissolved in 1 L of deionized water while being heated and continuously mixed. The solution was boiled for 1 min and then autoclaved at 121 °C for 1 h.

Biofilm suspension obtained from control slides was subjected to a four-fold dilution by combining 1 mL BS with 3 mL sterile phosphate buffer solution. For the BS obtained from UEGs, no dilution was performed. A total of 1 mL of each diluted/un-diluted suspension was added to a sterile Petri dish, followed by the addition of 20 mL of liquified marine medium acclimatized at 48±2 °C. The plates were thoroughly swirled to ensure proper mixing of the liquid medium with the sample and were left undisturbed in a biosafety hood until the medium was completely solidified. Incubation of the plates was carried out at 37±1 °C for 24 h, and the colony forming units (CFU) per cm² were recorded as the resulting data. All opaque growth spots were counted as a colony. Statistical consistency between dilutions indicated a lack of contamination.

Live/Dead analysis: To evaluate the viability of bacterial populations based on cell membrane integrity, the LIVE/DEAD BacLight™ Bacterial Viability Kit (Invitrogen, USA) was utilized. This kit enables the differentiation of cells with compromised membranes, with dead or dying cells stained red and cells with intact membranes staining green. Sample preparation and staining procedures followed the instructions provided with the BacLight™ Kit.

Initially, 6 mL of the biofilm suspension was transferred into a sterile centrifuge tube and centrifuged at 10,000×g for 10 minutes. The supernatant was carefully removed, and the resulting pellet was resuspended in a 20 mL buffer solution. After vortexing for 1 min, the suspension was incubated at room temperature for 1h. Subsequently, the suspension was centrifuged at 10,000×g for 10 minutes, and the pellet was resuspended in another 20 mL buffer solution. Once again, the sample was vortexed and centrifuged as described above. No dilution occurred during washing.

The harvested cells were suspended in a 2 mL buffer solution and vortexed for 1 min. From this suspension, 1 mL of the sample was mixed with 3 μL of the LIVE/DEAD BacLight Kit mixture containing a 1:1 ratio of SYTO™ 9 nucleic acid (final concentration: 29.91 μM) and propidium iodide (PI) (final concentration: 4.99 μM) and incubated in the dark for 15 minutes at room temperature. To visualize the stained bacterial cells and determine their viability, 5 μL of the stained bacterial suspension was pipetted onto a microscope slide and covered with a coverslip. Using a fluorescence microscope (AMG EVOS FL, USA), images were captured from eight randomly selected areas of each microscope slide. Green fluorescent protein (GFP) images were used to count live images, and red fluorescent protein (RFP) images were used to count dead cells. The number of live and dead cells was recorded, and the mean values were reported as the final result.

Protein and DNA analysis: For the quantification of unbound proteins, 3 mL of the biofilm suspension was subjected to centrifugation (10,000×g for 10 minutes). The resulting supernatant was collected to measure the concentration of unbound proteins. The pellet obtained from centrifugation was resuspended in 1 ml of phosphate buffer solution. A10 UL aliquot of the suspension was used to quantify the total bound and intracellular proteins. The quantification of total proteins in each sample fraction was performed using Qubit™ Protein Assay Kit (Invitrogen, USA) and a Qubit R 2.0 Fluorometer without protein isolation.

To extract the total DNA concentration from biofilm samples, the QIAamp Viral RNA Kit (Qiagen, USA) was used. The QIAamp Viral RNA Mini Kit can extract small quantities of viral RNA and cellular DNA If both are present in the sample. The biofilm suspension (900 μL) was concentrated by centrifugation at 10,000×g for 10 minutes. Supernatant was decanted. Phosphate buffer solution was added to the pellet to a final volume of 140 μL. The subsequent extraction by the QIAamp Viral RNA Kit was done according to the manufacturers protocol. Quantification of DNA was conducted using Qubit™ 1× dsDNA HS (High Sensitivity) Assay Kit (Invitrogen, USA) and Qubit R 2.0 Fluorometer.

### Example 4: Results

### Coating Thickness

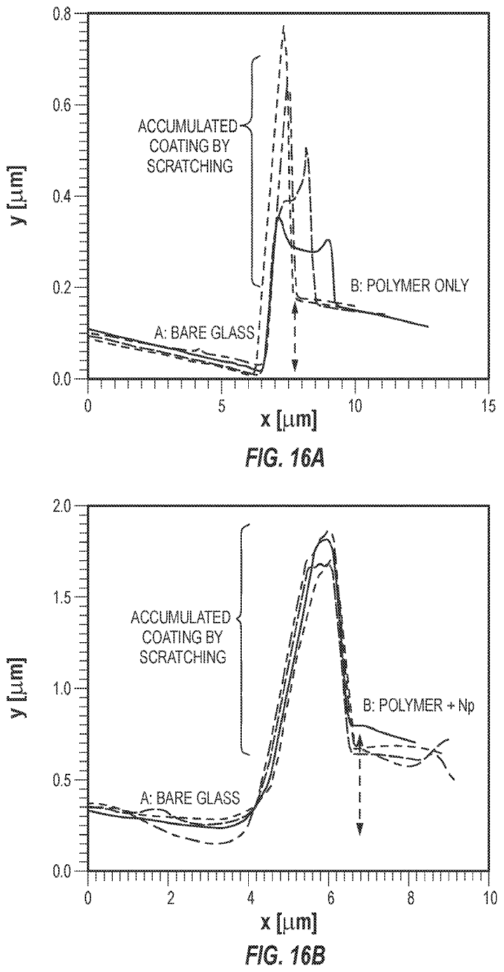

The AFM analysis of the glass surfaces coated with only polymer and nanoparticles+polymer was performed to measure the mean thickness of the coating material. FIGS. 15A-15B show top view images of glass surface coated with only polymer (FIG. 15A (a₁) and nanoparticles+polymer (FIG. 15B (b₁) as well as the respective corresponding cross-sectional profiles (FIG. 16A (a₂) and FIG. 16B (b₂). As illustrated by the peak in each image, the method of scratching is used in analyzing the polymer thickness leaves accumulated residue. Therefore, the reported polymer thickness is the measurement after the residue (test) minus the measurement before the residue (glass). Based on the cross-sectional profiles, the mean thickness of only polymer and nanoparticles+polymer was 151±9 nm and 422±83 nm, respectively.

In this example, the goal was to minimize the polymer thickness applied to the substrate while ensuring complete coverage of the substrate and nanoparticles. The polymer thickness should not have a strong effect on the UV performance of the UEGs due to the high transparency of >95% for 1 mm thickness of material. However, the thickness and method of polymer application will affect both the durability and cost of the UEGs.

Scattering nanoparticles enable glasses to emit UVC light along the entire surface: To obtain effective UVC emission across their surface, UEGs were fabricated using 200 nm silica spheres as scattering centers. A previous study demonstrated that silica spheres>200 nm coated optical fibers resulted in a UV emission of ~4.4 μW/cm². This value was slightly higher than emission by 100 nm diameter particles (3.4 μW/cm²) and greater than 5 times higher than the 50 nm spheres.

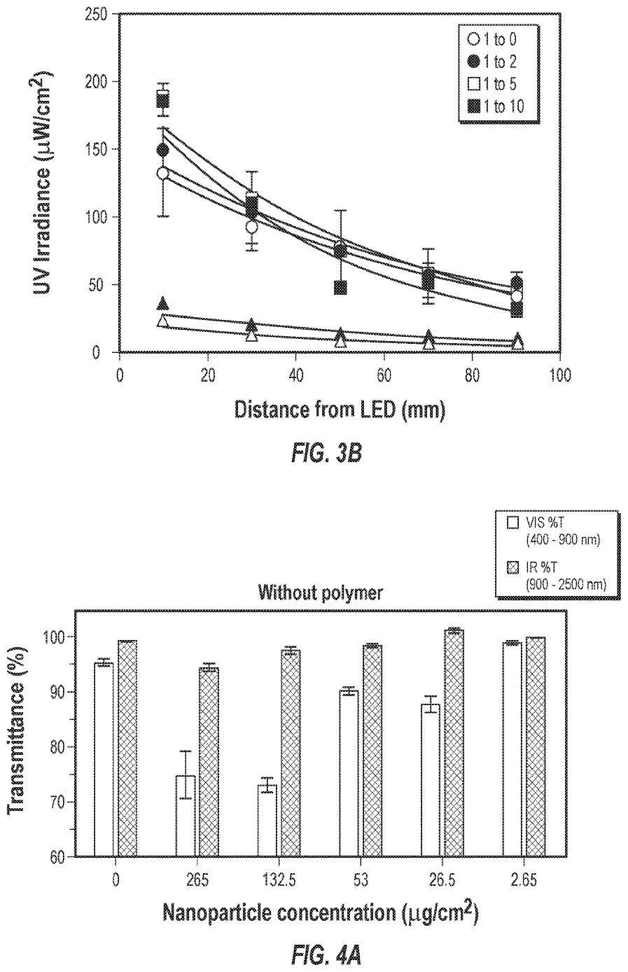

Referring now to FIGS. 3A-3B, the UV irradiance profile through the length of the slides coated with different concentrations of nanoparticles without (FIG. 3A) with and a secondary polymer coating (FIG. 3B). In a homogeneous longitudinal waveguide, the intensity of the scattering light follows an exponential decrease (EQ. 1):

Iᵣ(z)=Iᵢe^{-az} (EQ. 1)

where Iᵣ(z) represents the intensity of light remaining in the waveguide at any axial position z; Iᵢ is the amount of light that enters the slide at the z=10 mm, and α is the attenuation coefficient. α is a function of the optical and physical properties of the glass and the surface coating. The attenuation coefficient is a metric for quantifying the decrease in light intensity resulting from the combined effect of scattering and absorption over a given unit length of travel in an attenuating medium (α=αₛ(scattering coefficient)+αₐ absorption coefficient). SiO₂ has low absorption at UVC range due to its insulation characteristics. The bandgap energy of falls between 7.52 and 9.6 eV. The photon energy of 4.67 eV in 265 nm wavelength cannot be absorbed and will not be able to excite enough of the valence band electrons to jump into the conduction band. Since most of the attenuation is a result of light being emitted from the glass, the same equation was used to represent the scattering coefficient of the glass slide (α=αₛ).

The scattering attenuation coefficients for each concentration of nanoparticle-coated slide were determined by exponentially fitting the scattering intensity profiles (FIGS. 3A-3B) and are presented in TABLE 1. As illustrated, the UV irradiances increase along the slide surface as the concentration of nanoparticles increases from 0 to 26.5 μg/cm². Higher concentrations led to an insignificant (p-value>0.05, paired t-test) change in UV scattering and scattering coefficient. Without wishing to be bound by any theory, it was assumed that scattering centers interact with the evanescent wave created at the core surface of the waveguide. Evanescent waves refer to the electromagnetic disturbance that occurs due to total internal reflection at the interface of a transmitting medium. The wave amplitude decreases exponentially with the distance from the interface. Higher concentration of nanoparticles would result in higher wave interaction. However, increasing the concentration of nanoparticles also results in more light refraction back towards the core of the waveguide instead of being emitted outwards. Therefore, slightly reducing the light that can be used in disinfection.

TABLE 1
Scattering coefficient values of slides coated with different concentrations
of nanoparticles, with and without polymer coating.

| Polymer | Dilution value of nanoparticles | | | | | |
|---|---|---|---|---|---|---|
| coating | 0 to 1 | 1 to 0 | 1 to 2 | 1 to 5 | 1 to 10 | 1 to 100 |
| Yes | 0.017 | 0.014 | 0.013 | 0.017 | 0.021 | 0.015 |
| No | 0.017 | 0.018 | 0.016 | 0.018 | 0.023 | 0.009 |

FIG. 3B illustrates the UVC profile after a polymer layer was added to the slides. There was no statistically significant variation in UV irradiance after polymer coating of UEGs prepared at 26.5 to 265 μg/cm² of nanoparticles. However, more than a 50% decrease in UV irradiance was observed for UEG prepared with a nanoparticle concentration of 2.65 μg/cm². Such decrement can result from decreased interaction between nanoparticle and glass slide after the polymer is introduced and was discernible at lower concentrations of nanoparticles. Glass slides were coated by electrostatic attachment positively charged aminated SiO₂ nanoparticles on the negatively charged glass surface. In this process, electrostatic interaction results in the proximity and attachment between the nanoparticle and the glass surface. The negatively charged polymer can weaken the interaction between glass and nanoparticles and, therefore, increase the distance between the two. This phenomenon would result in a decrease in UV emission after polymer application. On the other hand, although CYTOP is an ultra-high UV transparent (over 95%) polymer, it can absorb a small amount of UV irradiation, contributing to the decrease in UV emission.

FIG. 5 depicts the UV irradiance profile and spectral output for the final configuration of UEGs. After waterproofing the case with silicone binder, the UV irradiance decreased by approximately 70% from its original output. Since the scattering coefficient remained unchanged (as =0.021), it can be concluded that the only change was in the light availability, likely due to the addition of silicone as a water barrier. Silicone has a higher index of refraction than glass slides. Therefore, when light encounters the silicone, it is refracted out of the slide.

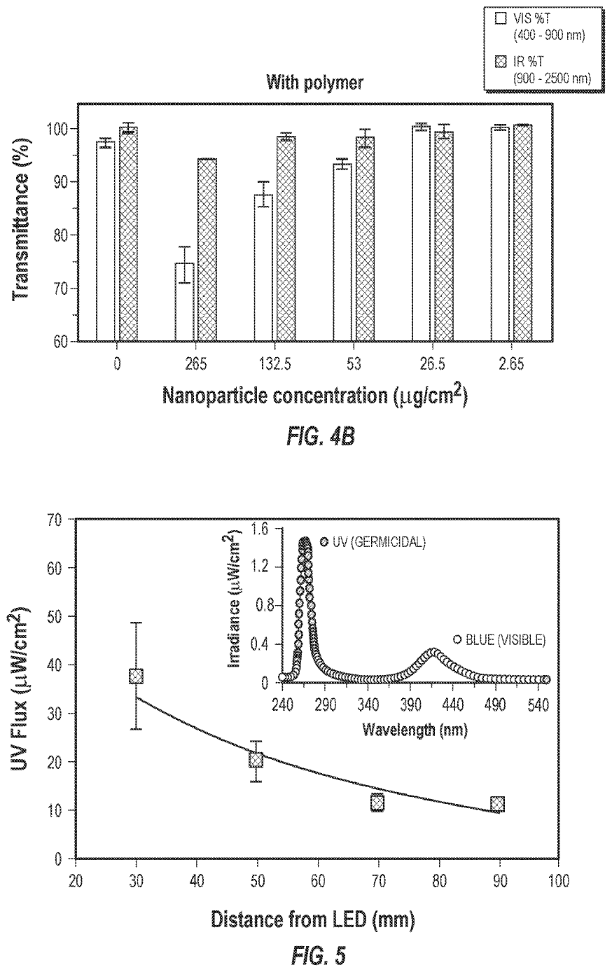

Nanoparticle concentration affects the optical transparency of the glass: The transparency of the coated slides was a crucial consideration in this work. FIGS. 4A-4B presents the Vis-NIR transmission spectra of glass slides that were coated with different concentrations of nanoparticles, both with and without polymer. In the Vis region, the slide coated with 2.65 μg/cm² of nanoparticles (dilution value=1:100) exhibited a transparency of over 95%, which was comparable with the transparency of the control slide. While Vis transparency was decreased by increasing the concentrations of nanoparticles from 26.5 to 265 μg/cm² (FIG. 4A). Since silica nanoparticles are transparent in the visible spectral region and do not absorb visible light, the decreased light transmission by increasing the silica nanoparticles concentration is attributed to the strong light scattering, including resonant and random scattering, on the surfaces of the silica nanoparticles19. Applying a transparent polymer coating onto the slides coated with 26.5, 53, and 132.5 μg/cm² of silica nanoparticles increased the light transmission at the visible region by 13, 3.3, and 16.6%, respectively (FIG. 4B). This increase in transparency upon polymer addition can be due to the decrease in the scattering of visible light. The Rayleigh theory describes the reduction of light intensity due to scattering in nanoparticle-polymer composites. This theory was expressed by Novak20 through EQ. 2:

$$\frac{I}{{I}_{i}} = {e}^{[ \frac{3 {V}_{p} x {r}^{3}}{4 {λ}^{4}} ( \frac{{n}_{p}}{{n}_{m}} - 1 ) ]} \qquad ( EQ . 2 )$$

where I represents the intensity of the transmitted light by the nanoparticle-polymer composite, V. and r are the volume fraction and radius of the particles, respectively. In this equation, x is the optical path length, while nₚ and nₘ represent the refractive index of nanoparticles and polymer matrix, respectively.

According to the above-mentioned equation, if nₚ=nₘ, the scattering tends to be zero, and the transmission is equal to 1 (T=100%). The intensity of scattered light by silica nanoparticles (nₚ=1.43) at the visible region can be decreased by a polymer matrix (nₘ=1.34 for CYTOP polymer), resulting in more transparency. However, polymer coating did not increase the transmission of the slides with concentrations of 2.65 and 265 μg/cm² of nanoparticles.

As indicated by Equation 2, increasing the thickness of the coating results in an extended optical path length, that can therefore lead to a reduction in transparency. We assume that, in general, thicker coatings would significantly decrease the UV transparency. However, because the polymer has >95% transparency for a path length of 1 mm, changes in UV emission due to polymer thickness were negligible. Every slide tested transmitted more light in the NIR region as compared to the Vis region. The light transmittance of the slides used for submersion (concentration of nanoparticles: 26.5 μg/cm²) in the Vis region was 88±1.6% without polymer coating and 100.4±0.6% with the polymer coating. Light transmission in the NIR region was 101±0.6% without polymer coating and 99.4±1.3% with polymer coating. Data exceeding 100% are attributed to experimental errors in the instrumentation. These results suggest that fabricated transparent UEG at 265 μg/cm² of nanoparticles also fit the near-infrared working window, making them suitable for applications like near-infrared cameras.

Resolving power is another important feature of transparent surfaces. This is the ability of an instrument or the human eye to distinctly differentiate between two objects positioned closer than the minimum angular separation discernible to the observer's eye. All tested slides, coated with different concentrations of nanoparticle and with or without polymer, exhibited 100% resolving power (versus blank) and excellent optical clarity (data are not shown).

### Example 5: Biofilm Prevention by UEG

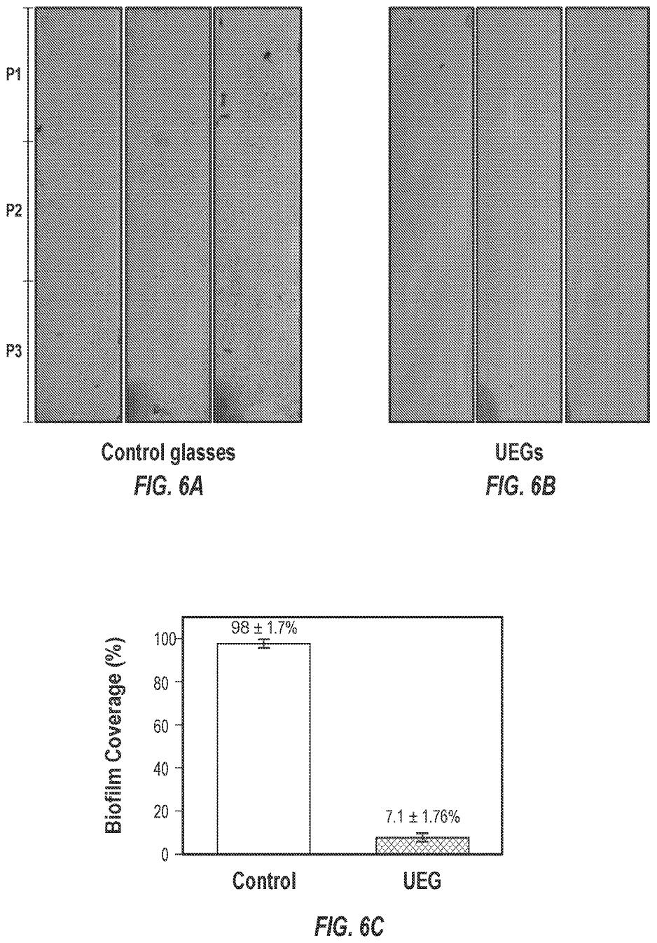

UEG decreases biofilm coverage: FIGS. 6A-68 illustrates the photographs of the control slides and UEGs that were exposed to continuous UVC radiation after being submerged for 20 days. Visual assessment results (FIG. 6C) indicated a reduction of over 92% in biofilm coverage on UEGs compared to the control slides. The slides modified with scattering centers delivered more than 7.7 times the UVC dose and achieved 13.7-fold less biofilm coverage compared to the control slides. Most of the growth was micro biofouling, green algae and amphipod tubes.

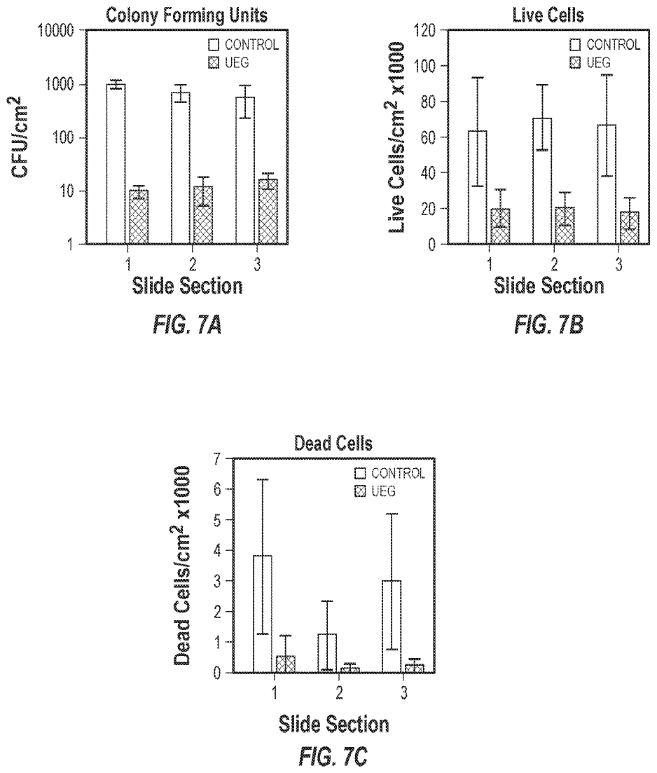

Quantification of biofilm growth developed on slides as CFUs are presented in FIGS. 7A-7C, The culturable cells attached to the surface of each slide were quantified as CFU and are presented in FIG. 7A. On average, the control slides exhibited CFU counts of about 1000±190, 700±280, and 600±310 CFU/cm² for p₁, p₂, and p₃ sections, respectively. In contrast, the UEGs showed significantly lower CFU counts, with only 10±2.7 CFU/cm² for p₁, 12±6.6 CFU/cm² for p₂, and 16±5.5 CFU/cm² for pa. Although a descending trend in CFU was expected with increasing the distance from the UVC source, no statistically significant (p-value>0.05, paired t-test) difference in biofilm abundance was observed among across three sections of the slides, suggesting proximity to the light source did not impact biofilm mitigation. The average UV dose for section p3 totaled approximately 19.56 J/cm² for 20 days of submersion, with an irradiance of 11.32 μW/cm² (FIG. 5). This dose and irradiance were highly effective in preventing biofilm attachment and growth on the surface resulting in 1.79 log reduction of biofilm in comparison to the control.

It has been reported that a minimum UVC dose ranging from 10 to 300 J/m² achieves a maximum inactivation (6 log inactivation) of planktonic bacteria. These values may vary considerably for surface disinfections. For example, a dose of 25 J/cm² was found to be effective in biofouling control and 1 log Inactivation of Navicula incerta cells on a UV-lucent silicone integrated with UVC LEDs. Torkzadeh et al. observed that a UV dose of 8.7 J/cm² at 254 nm achieves 1.3 log inactivation (95% decrease in E. coli surface biovolume), while a 0.02 J/cm² dose was sufficient for approximately 5-log inactivation of planktonic E. coli. Bacteria in biofilms are physiologically and morphologically different from their planktonic growing counterparts and, once they are attached to the surface become highly resistant to disinfection. Additionally, marine bacteria, due to their aggressive biofilm-forming lifestyle and adaptation to harsh oceanic, may require more intensive irradiation for effective biofilm control. On the other hand, the accumulation of sediment and organic materials on the surfaces can act as a shield for biofilms exposed to UV irradiation, affecting UV transmission. Therefore, a higher UV intensity would be required for the effective removal of the biofilms formed in the marine environment.

Lower viable cell count in UEG: The LIVE/DEAD BacLight Bacterial Viability Kit was employed to assess the viability and distribution of live and dead cells within the biofilm samples, using membrane integrity as an indicator for cell viability. Viable cells are assumed to have intact and impermeable cell membranes that prevent the penetration of certain staining compounds, whereas dead cells are recognized by their disrupted or broken membranes.

Microscopy analysis of the samples revealed the presence of microcolonies and individual cells within the biofilm samples. A small number of dead cells were observed, along with amorphous red propidium iodide-stained material, which may represent DNE-containing debris from lysed cells.

In the biofilm grown on the UEGs, the number of cells exhibiting both live and dead fluorescence was limited, and these cells were sporadically distributed. Live and dead cells were counted separately for the control slides and UEGs and the results are illustrated in FIGS. 7B-7C, respectively.

Comparable to the results obtained from the pour-plate method, no significant difference (p-value>0.05, pared-t-test) in the number of live/dead cells was observed among different sections of the slides. On average, the number of live bacterial cells in the biofilm grown on the UEGs was found to be 73±2.1% lower than that on control slides. The number and proportion of dead cells in biofilm grown on UEG was also lower than that of control slides (FIG. 7C).

In addition, the live: dead ratio was 82:1 and 25:1 for biofilm grown on UEG and control slides, respectively. This can result from the mode of action of the LIVE/DEAD Baclight stains being incongruous with the UV inactivation mechanism. The LIVE/DEAD BacLight method allows for the detection of cell states beyond just live and dead cells. This includes the identification of live injured cells that are unable to grow on agar plates. Germicidal UVC prevents the buildup of bacterial biofilms by breaking the chemical bonds between DNA and RNA polymers within microorganisms, disrupting the genetic code, and preventing DNA from replicating. However, since UVC does not directly affect membrane integrity, many metabolically inactivated cells did not stain red, as their membranes were still intact. Consequently, the number of live cells estimated using this method is noticeably greater than the counts obtained from the pour-plate method. It can be concluded that the pour plate method can be a more accurate technique for quantifying the viable bacteria in UV-irradiated biofilm.

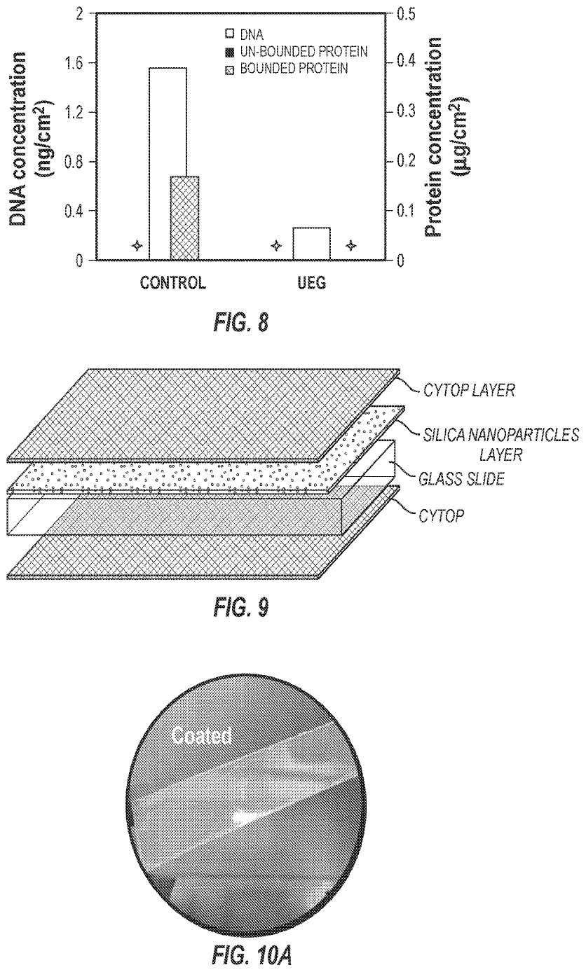

Protein and DNA analysis: DNA and proteins are two of the main molecules identified in natural marine biofilms that are essential for biofilm growth and survival. The total DNA unbound and bound protein concentrations were determined on each UEG and control slide and are illustrated in FIG. 8. The mean value of total DNA concentration for control and UEG slides were 1.6±0.33 ng/cm² and 0.2±0.1 ng/cm², respectively. These results align with the number of CFU observed in both control and UEG slides. UVC radiation particularly targets nucleic acid molecules, and it interacts with bacterial DNA to inhibit growth, reproduction, and the formation of biofilms. It is worth noting that an increased cell density in the biofilm corresponds to a greater concentration of DNA. The concentration of bound proteins in the control slides was 0.17±0.05 μg/cm². However, bound protein concentration was below detection in the UEGs. Additionally, the concentration of unbound proteins was below detection on all slides. It is likely that the reconstituted concentration of proteins in these samples fell below the detection limit of the method employed. It should be noted that UVC has been reported to irreversibly damage proteins and prevent enzymatic DNA repair processes. Proteins are a crucial component of extracellular polymeric substances (EPS) and, together with polysaccharides, play a vital role in the structural properties of biofilms due to their gel-like nature.

The disclosed examples illustrate the potential of employing UEGs for effective disinfection and biofilm prevention. Through meticulous examination, it was determined that the concentration of nanoparticles plays a pivotal role in attenuation and scattering UVC light, with the optimal concentration of 26.5 g/cm² identified to maximize UV irradiance intensity while preserving acceptable transparency. The scattering nanoparticle-coated glass surfaces exhibited remarkable inhibitory effects on biofilm growth, surpassing control slides by reducing biofilm coverage by over 98% (based on the number of CFUs) during 20 days of marine submersion. Detailed microscopic analysis also revealed lower viable cell counts and a decreased proportion of dead cells within the biofilm growth on the modified surfaces. These results not only underscore the potential for UEGs to enhance surface hygiene and mitigate microbial contamination but also open new avenues for their utilization in diverse disinfection applications. In addition to the applications in marine vessels, the described UEG can be used for disinfection of transparent surfaces such as windows of flotation spheres and moored buoys, camera lenses, and sensors for oceanographical, agricultural, water treatment, and process design applications. UEGs can be manufactured on a commercial scale, considering the surrounding materials to be UV-resistant. Overall, this study contributes valuable insights into the field of surface engineering and sets the stage for further advancement in the realm of antimicrobial coatings and treatments.

### Example 6: UV Emitting Transparent Surface for Biofilm Prevention

In this example, a study was conducted to test the glow of UV light from the inside of glass on preventing biological attachment and growth. Disturbing the electromagnetic wave by adding scattering nanoparticles onto the glass surface caused the light to leak out of the waveguide substrate, resulting in side-emission and glowing effects.

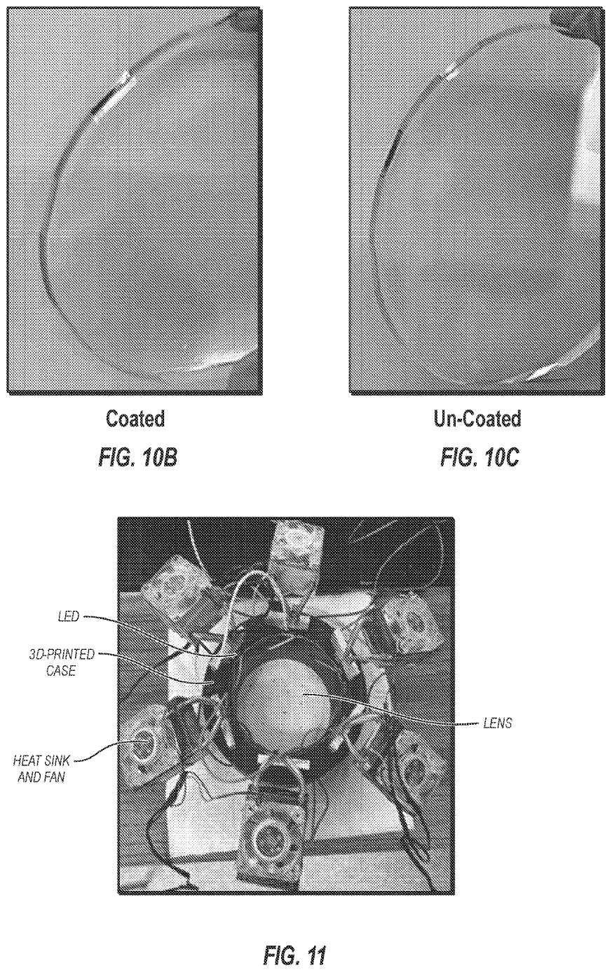

UV-emitting glass (UEG) slides and lenses were fabricated by first dipping the slides in acetone for cleaning. SiO₂ nanoparticles were then coated onto the slides by an electrostatic deposition mechanism. Finally, the slides were coated with a UV-transparent polymer. FIG. 9 shows a schematic of each layer of the slides. FIGS. 10A-10C shows photographs of the slides before and after coating.

The UV emission profiles of the UEG lenses can be measured at multiple wavelengths. FIG. 11 shows the UV measurement setup for a 100 mm (diameter)×6 mm (thickness) lens.

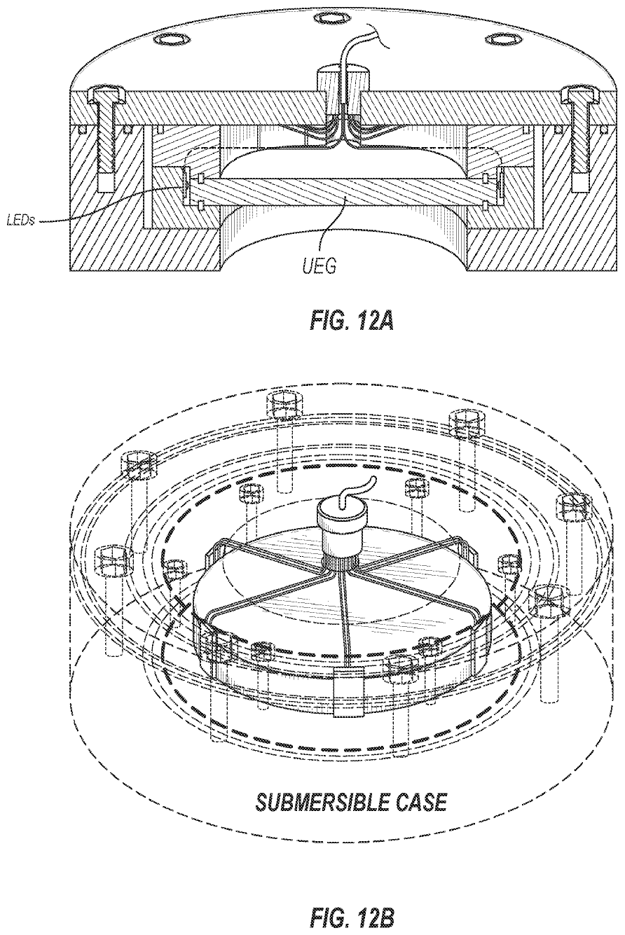

FIG. 12 shows an example case design for the submersion of UEGs in a marine environment.

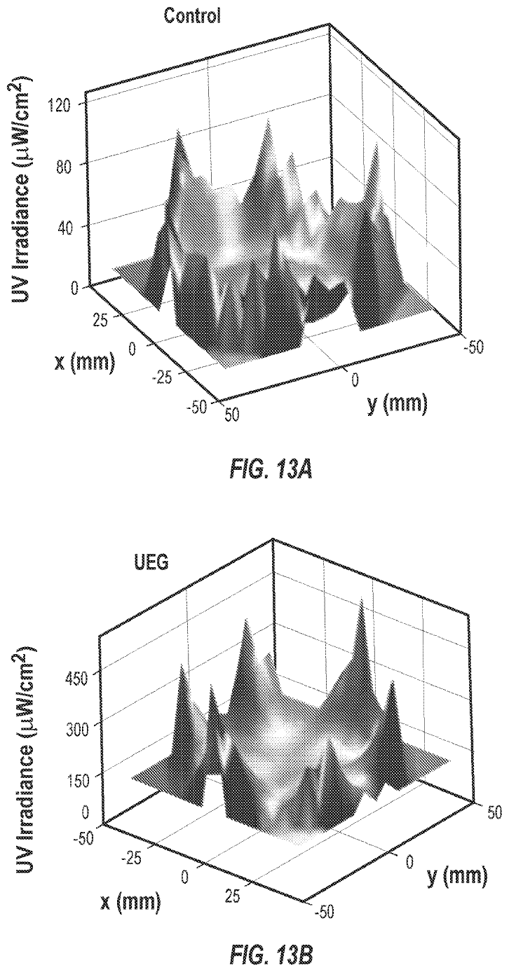

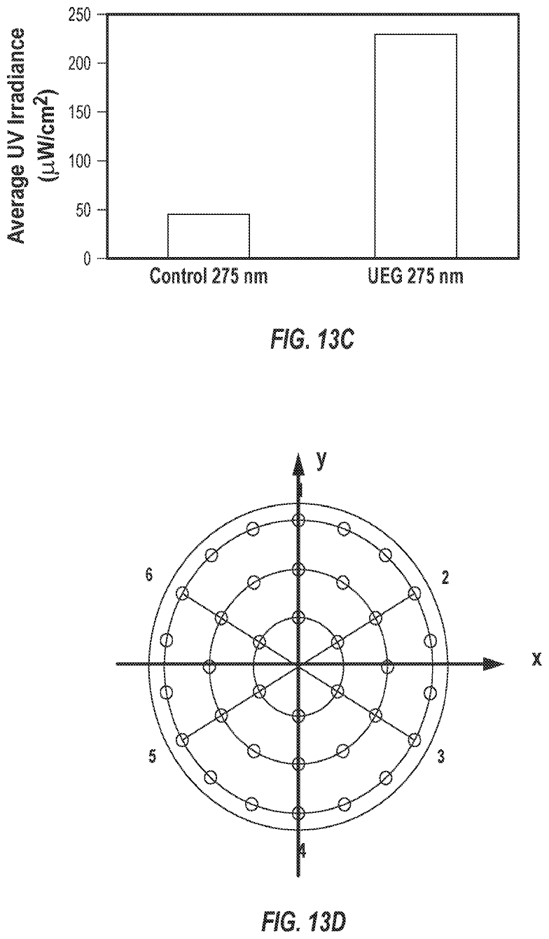

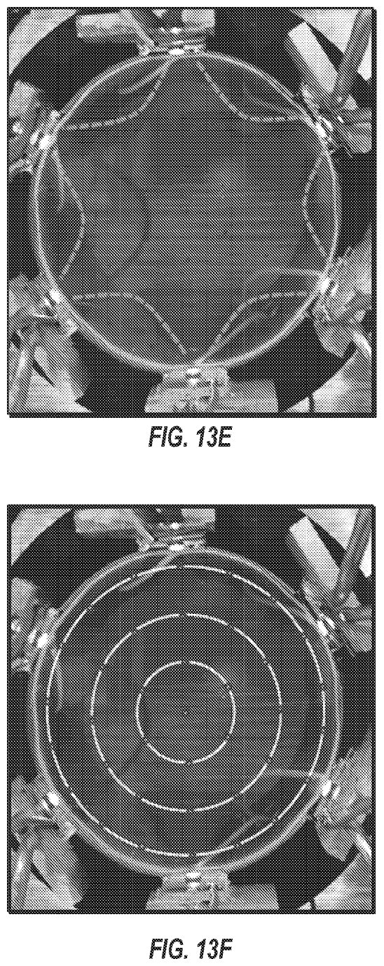

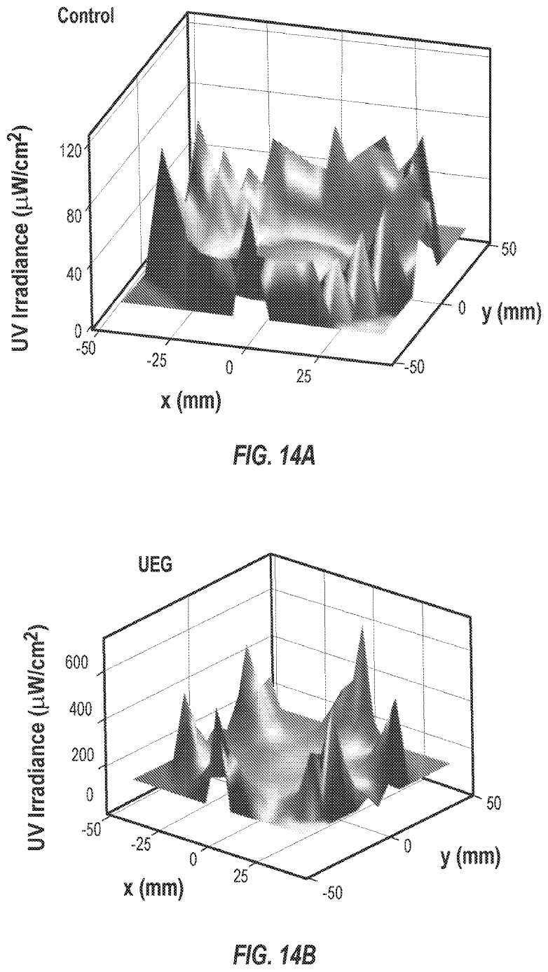

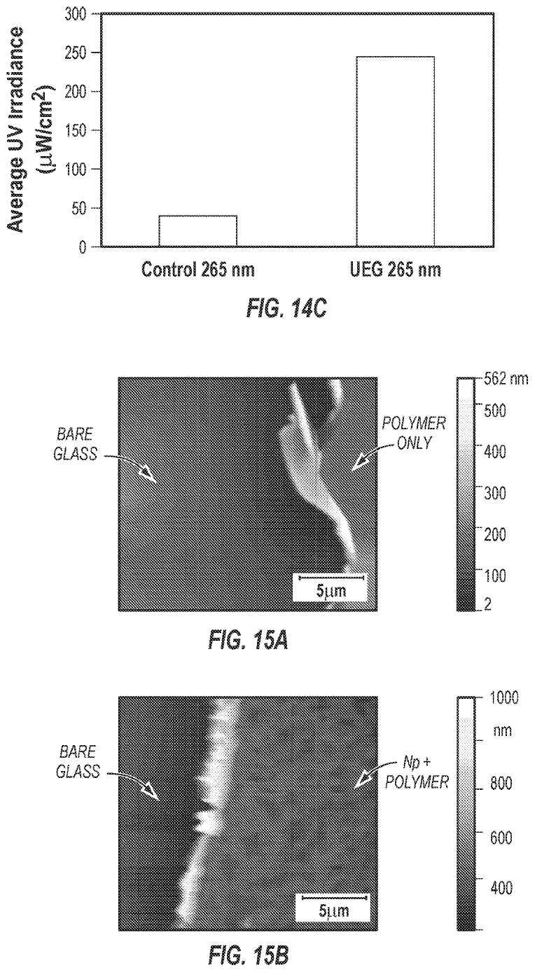

FIGS. 13A-13F show the UV emission profiles of UEG at 275 nm. FIGS. 13E-13F, for example, show the light propagation from 6 different LEDs positioned along the circumference of the lens. FIGS. 14A-14C show the UV emission profiles of UEG at 265 nm. The UEG used to generate both emission profiles was a 100 mm (diameter)×6 mm (thickness) lens.

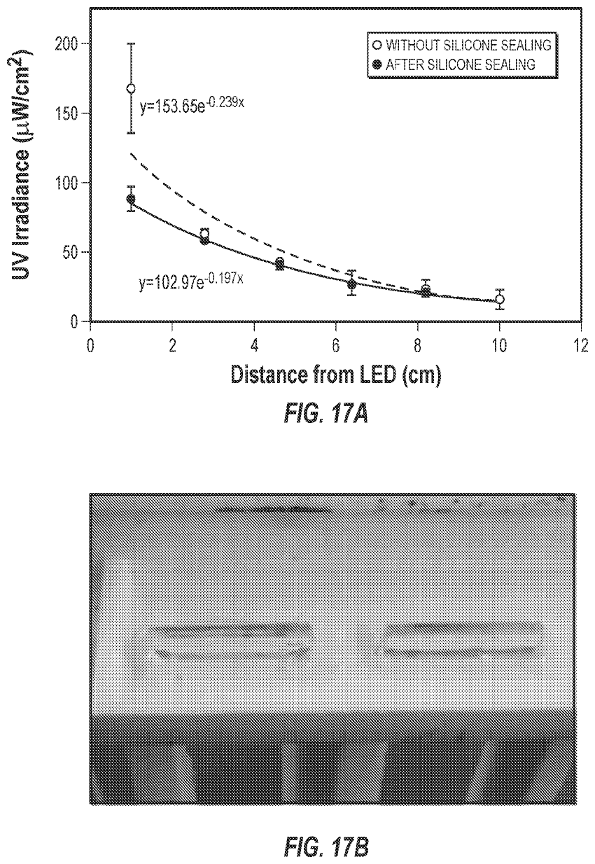

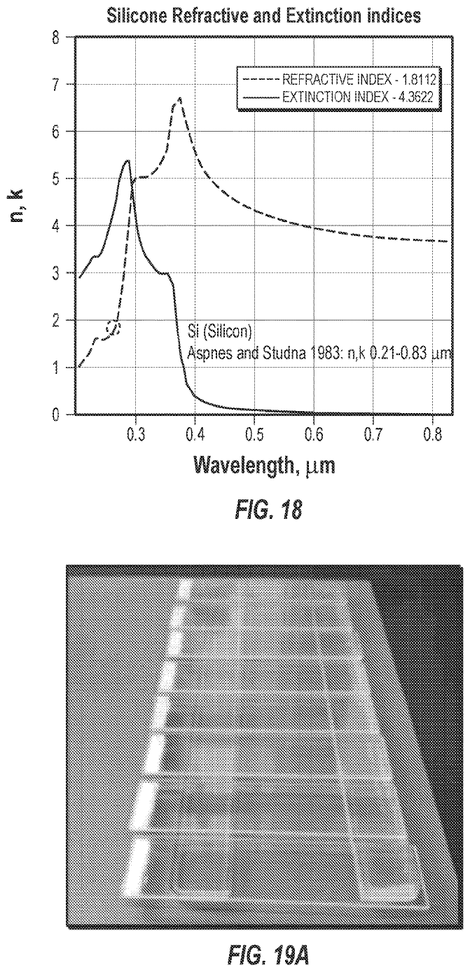

FIGS. 17A-17B and TABLE 2 demonstrate that silicone sealing decreases UV irradiance from glass slides. FIG. 18 shows that the materials with high absorption coefficients have high refractive indices.

TABLE 2
Silicone sealing decreases UV irradiance from glass slides.

| Distance | Before silicone sealing | After silicone sealing |
|---|---|---|
| (cm) | (μW/cm²) | (μW/cm²) |
| 1 | 167.515 | 87.755 |
| 2.8 | 63.61 | 59.355 |
| 4.6 | 43.39 | 41.205 |
| 6.4 | 27.605 | 26.02 |
| 8.2 | 23.69 | 21.595 |
| 10 | 16.315 | 14.685 |

This study used low refractive index-polymer to resolve the negative effect of silicone sealing on UV irradiance from glass slides. For this purpose, polytetrafluoroethylene (PTFE) with a refractive index of 1.356 was used. For coating the slides, the following procedure was followed:

1) The glass slide was coated with silica nanoparticles using the same method described previously. Coated slides are allowed to be dried for 2 h at room temperature.

2) To apply a PTFE layer coating, 60 μL of PTFE was pipetted onto the top 0.6 cm section of the nano-coated slide's surface, precisely targeting the area that is intended to be covered with silicone sealant. The total surface area of the slide coated with PTFE was 1.44 cm². To speed up the drying process, indirect heating was applied using a hot plate.

3) The slides were coated with CYTOP polymer using the same method as described previously. Due to the PTFE layer's easy removability, the CYTOP polymer coating was carefully applied in a bottom-to-top direction, preventing any potential scrubbing or dragging of the PTFE on the slide surface. After coating, the slides were left to dry overnight.

4) The back side of the slide was coated with a PTFE layer by pipetting 40 μL of polymer onto the top 0.6 cm section of the bare surface and dried by indirect heating.

5) The back side of the slide from step 4 was coated with CYTOP polymer according to the method outlined in step 4.

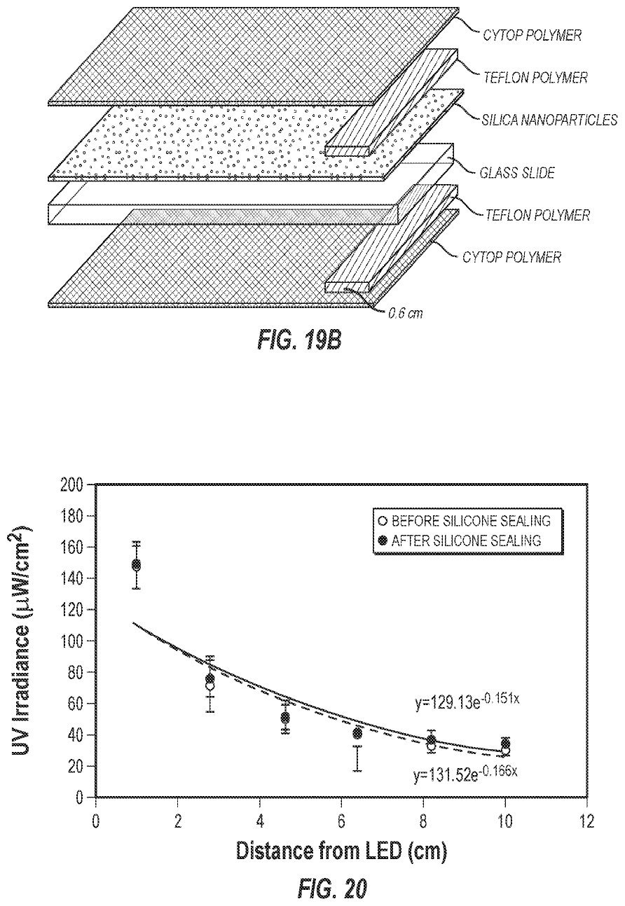

The PTFE coating is highly efficient in preventing UV light from being absorbed by the silicone sealant. FIGS. 19A-19B depict the PTFE-coated slides. FIG. 20 and TABLE 3 demonstrate that the PTFE coating omitted the adverse effect of silicon on UV irradiance.

TABLE 3
Effect of PTFE coating on UV irradiance.

| Distance | Before silicone sealing | After silicone sealing |
|---|---|---|
| (cm) | (μW/cm²) | (μW/cm²) |
| 1 | 146.51 | 148/11 |
| 2.8 | 72.32 | 76.09 |
| 4.6 | 51.32 | 51.83 |
| 6.4 | 39.99 | 41.04 |
| 8.2 | 32.49 | 37.17 |
| 10 | 30.83 | 35.54 |

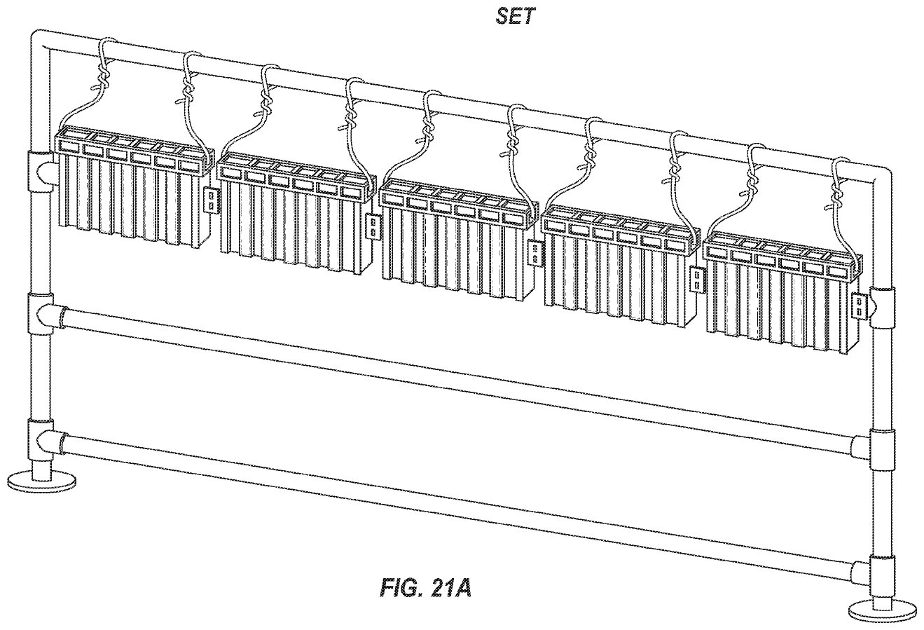

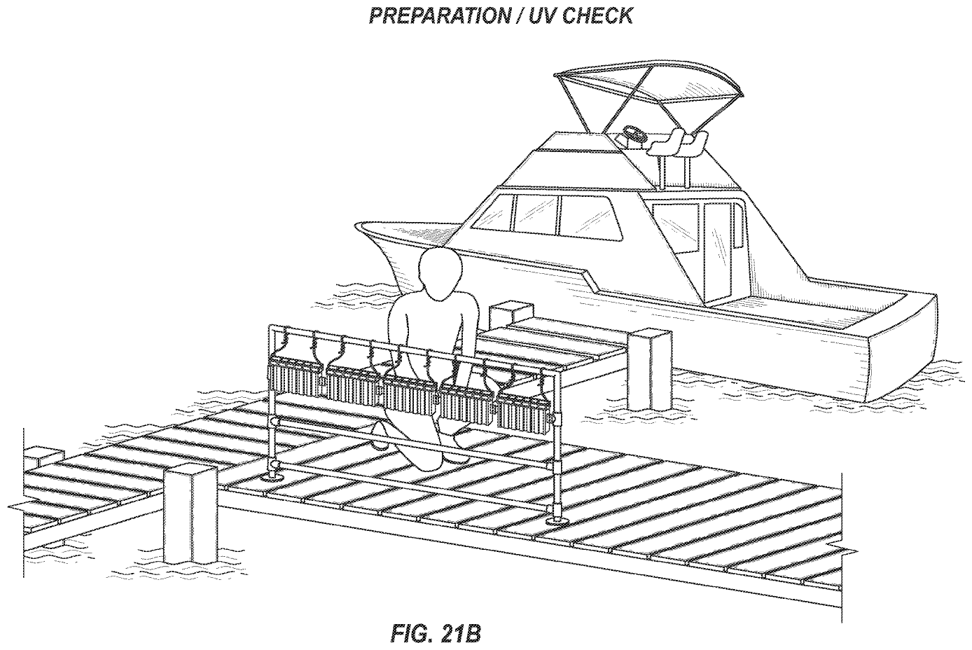

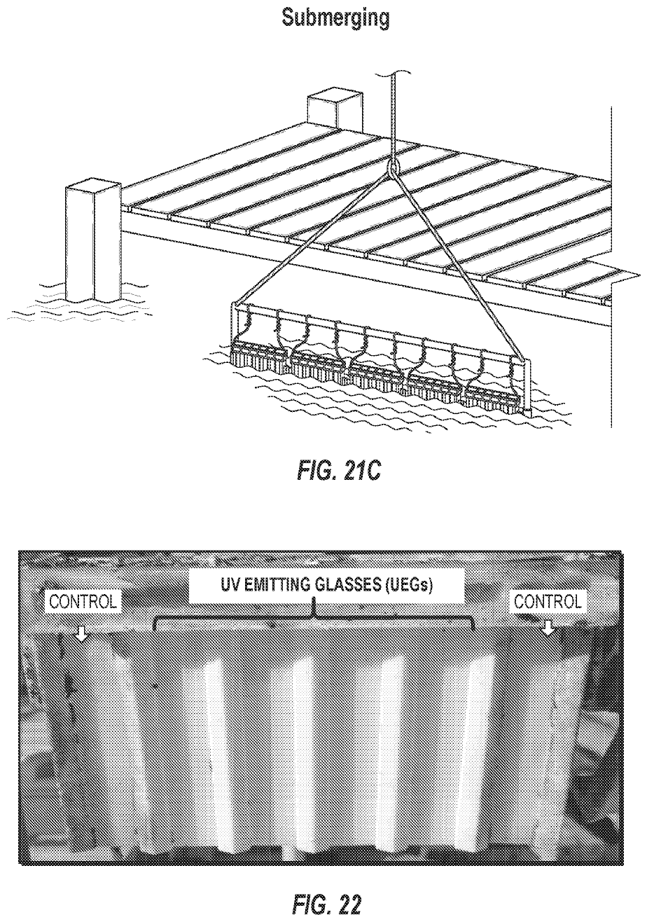

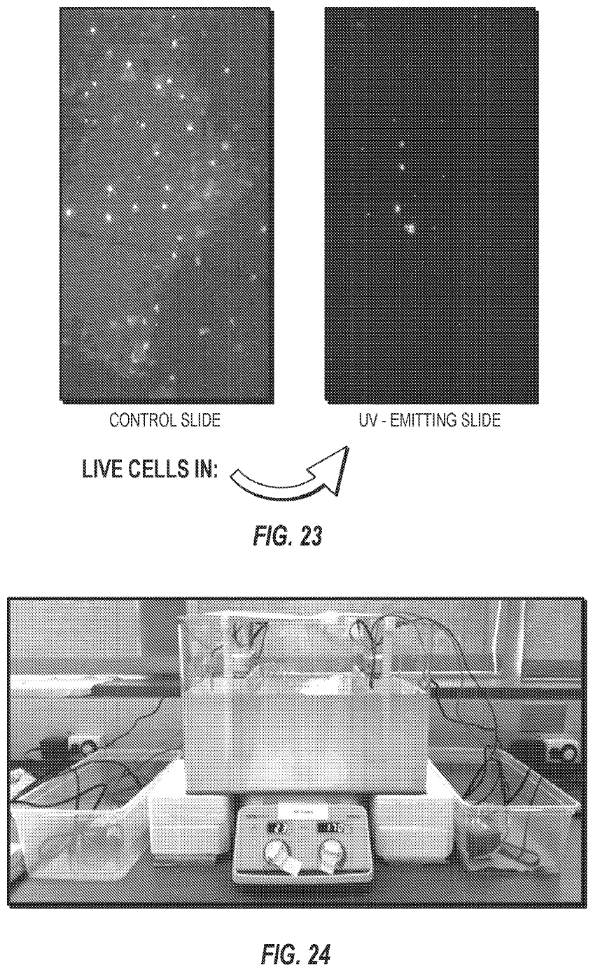

The study next tested the submersion of the UEGs in a marine environment. Cases were submerged at Port Canaveral, Florida, United States, for 20 days. FIGS. 21A-21C show the experimental setup. The cases used a UV input power of 16.8 W, a UV wavelength of 265 nm, and continuous irradiation mode. FIG. 22 shows a significant inhibitory effect (92%) of UEGs on the biofilm growth based on visual assessment. Overall, there was a 98.28% decrease in biofilm coverage based on CFU, as shown in FIG. 23.

Next, the study tested the submersion of the UEGs in laboratory conditions. The UEGs were submerged with either Pseudomonas aeruginosa or mixed marine bacteria for 8 days at a temperature of 16-19 °C. The UEGs used UV wavelengths of 265 nm, 280 nm, 310 nm, and 365 nm and continuous irradiation mode. FIG. 24 shows the experimental setup.

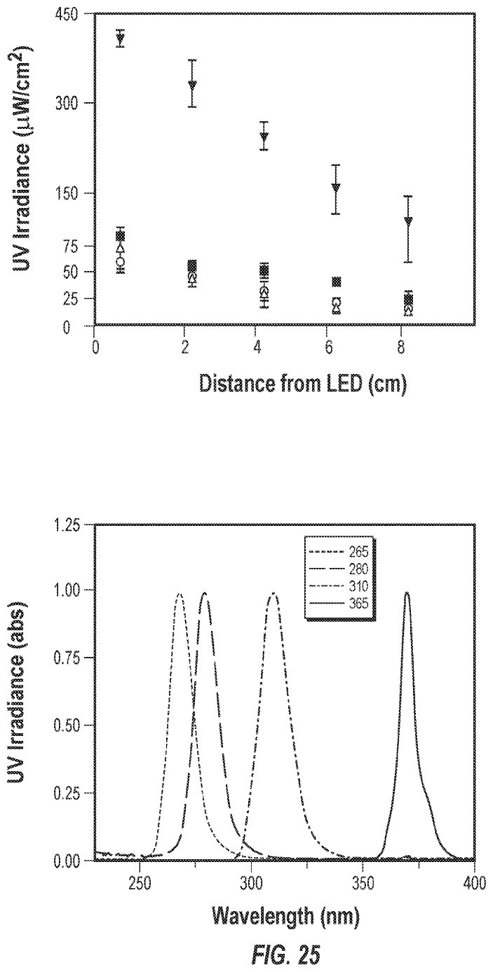

FIG. 25 shows the UV emission profiles of UEG slides at multiple wavelengths. The slides used were 24 mm (W)×110 mm (L)×2 mm (T).

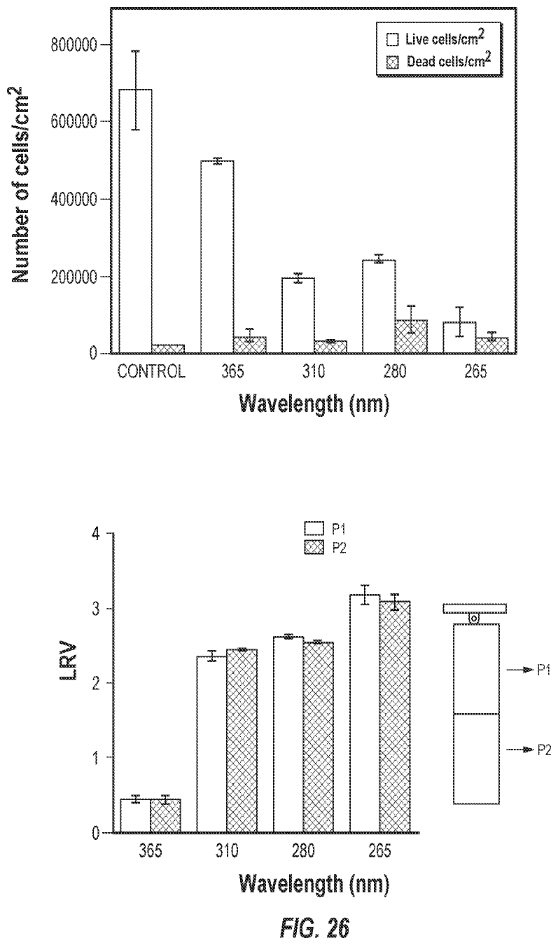

FIG. 26 shows the results of UEGs submerged with Pseudomonas bacteria. The highest Log Reduction Value (LRV) was observed at 265 nm. The wavelength effectiveness trend was: 265 nm>280 nm>310 nm>365 nm. There was no statistically significant difference between P₁ and P₂ in LRVs (verified by t-test). 310 nm showed only a 4% decrease in LRV compared to 280 nm.

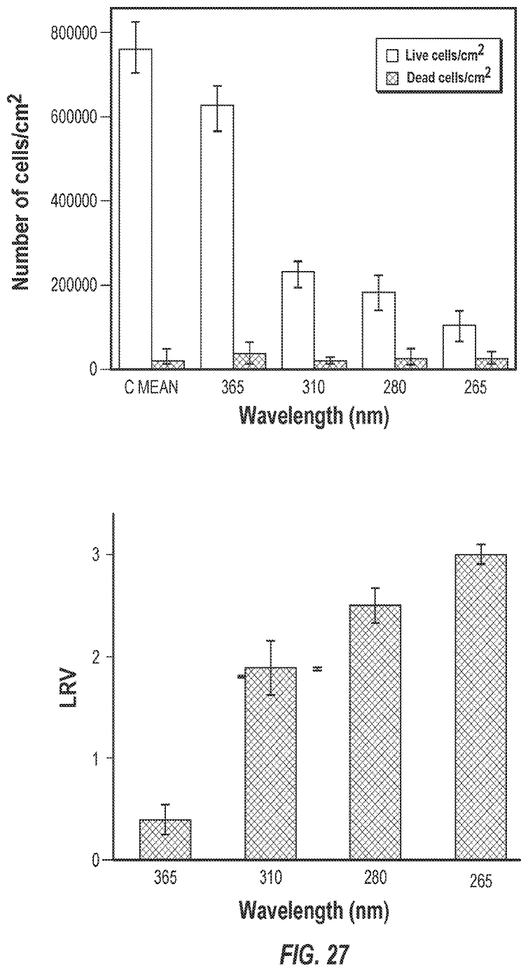

FIG. 27 shows the results of UEGs submerged with mixed marine bacteria. 265 nm showed the highest LRV of 3.04. The wavelength effectiveness trend was: 265 nm>280 nm>310 nm>365 nm. Viable cell count decreased from control slides to UEGs. This is an indication of biofilm prevention by cell detachment.

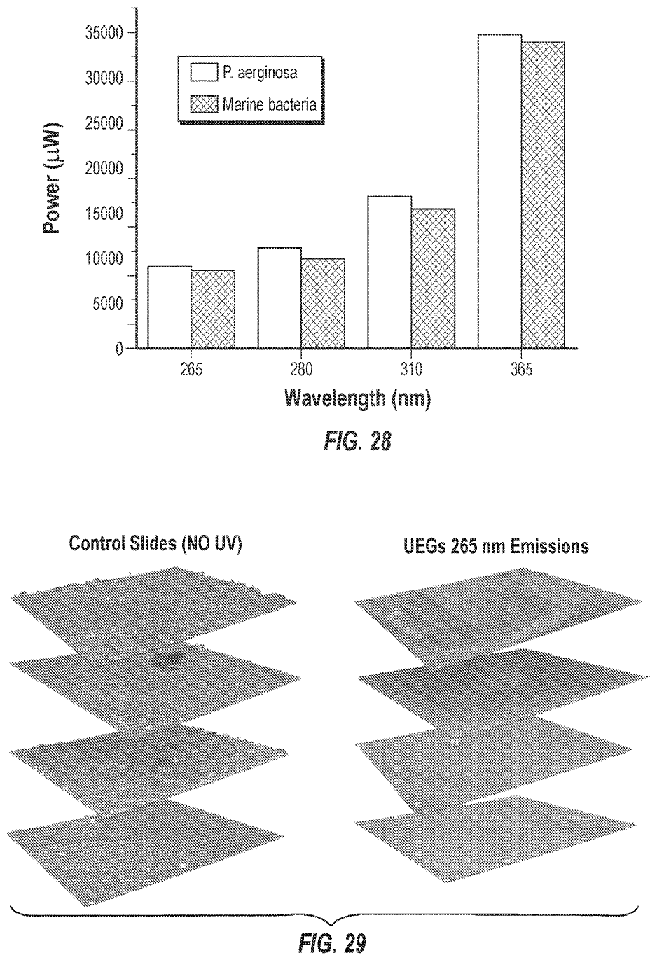

FIG. 28 shows electrical power per order. Understanding the electrical energy requirement is crucial for any light-based technology. The electrical energy requirement was calculated by:

$${EP}_{N} = \frac{{F}_{N} \times A}{C}$$

where EP_{N} represents the electrical power required to achieve 1 log reduction per square centimeter of surface, F_{N} represents irradiance (μW/cm²), A represents the area of the UEGs (cm²), and C represents wall plug efficiency. Overall, UVC and UVB showed lesser power consumption to get 1 LRV.

FIG. 29 shows optical coherence tomography (OCT) images of biofilm. The model used was GAN600, Ganymede series. The axial resolution was 3 μm, the prob was ProbTemplate_OCTG.ini, the scanning system was “Standard (OCTG),” and the nominal center wavelength was 905 nm.

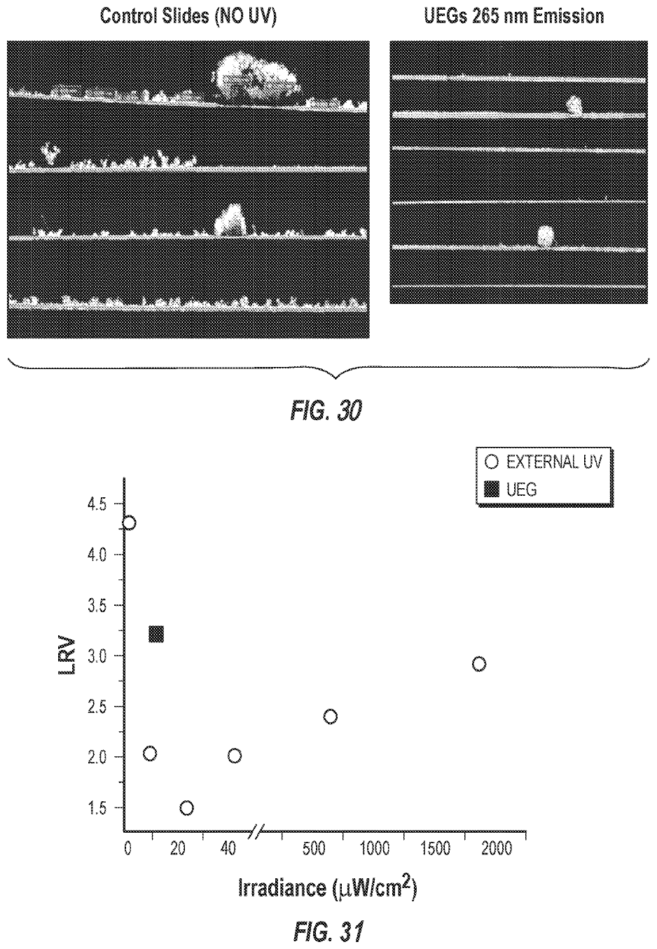

FIG. 30 shows measuring biofilm thickness at the areas covered by biofilm. The thickness variation was high (0 to 150 μm in UEG, 0 to 298 μm in control slides). FIG. 31 compares the effectiveness of UEG with external UV irradiation. Among these studies, UEG showed the highest LRV with minimum UV irradiance value. Only one other study showed the highest LRV with lower irradiance than UEG (Bak, J.; Ladefoged, S. D.; Tvede, M.; Begovic, T.; Gregersen, A. Disinfection of Pseudomonas Aeruginosa Biofilm Contaminated Tube Lumens with Ultraviolet C Light Emitting Diodes. Biofouling 2010, 26 (1), 31-38.)

These UEGs can be used in a variety of potential applications, for example: windows of ships, flotation spheres, and moored buoys; camera lenses and sensors; and water treatment and process design applications.

### Exemplary Aspects

In view of the described processes and compositions, hereinbelow are described certain more particularly described aspects of the disclosures. These particularly recited aspects should not, however, be interpreted to have any limiting effect on any different claims containing different or more general teachings described herein or that the “particular” aspects are somehow limited in some way other than the inherent meanings of the composites and formulas literally used therein.

Example 1: An article comprising (a) a light-emitting layer; and (b) one or more UV light sources coupled within the article such that a light beam is side-emitted by the light-emitting layer in a direction substantially perpendicular to a direction of the light beam; wherein the light-emitting layer comprises a plurality of light-scattering elements and exhibits at least 90% transmittance.

Example 2; The article of any examples herein, particularly Example 1, wherein the plurality of light-scattering elements comprise a plurality of nanoparticles.

Example 3: The article of any examples herein, particularly Example 1 or 2, wherein the one or more UV light sources are optically coupled with the light-emitting layer.

Example 4: The article of any examples herein, particularly Examples 1-3, wherein the plurality of light-scattering elements comprise silica nanoparticles having a size of 50 nm to 500 nm.

Example 5: The article of any examples herein, particularly Examples 1-4, wherein the light-emitting layer is positioned on a transparent or non-transparent substrate.

Example 6: The article of any examples herein, particularly Example 5, wherein the substrate is transparent, and the light-emitting layer is (i) disposed on a surface of the substrate, and/or (ii) at least partially embedded into a body of the transparent substrate adjacent to the surface of the substrate and wherein the plurality of light-scattering elements comprise one or more of a plurality of particles, defects, air bubbles, or a combination thereof.

Example 7: The article of any examples herein, particularly Example 5 or 6, wherein the one or more UV light sources are optically coupled with the transparent substrate and/or the light-emitting layer.

Example 8: The article of any examples herein, particularly Example 7, wherein the transparent substrate behaves as an optical waveguide such that the light beam propagates along a longitudinal axis of the transparent substrate and wherein the transparent substrate has a thickness of 250 microns to 50 cm.

Example 9: The article of any examples herein, particularly Examples 1-8, wherein the one or more UV light sources operate at the same or different wavelengths.

Example 10: The article of any examples herein, particularly Examples 1-9, wherein the one or more UV light sources comprise a light emitting diode (LED).

Example 11: The article of any examples herein, particularly Examples 1-10, wherein the light-emitting layer has a thickness of 50 nm to 5 cm.

Example 12: The article of any examples herein, particularly Examples 5-11, wherein the substrate has a regular or irregular geometry.

Example 13: The article of any examples herein, particularly Examples 5-12, wherein at least a portion of the light-emitting layer substantially conforms to at least one surface of the substrate.

Example 14: The article of any examples herein, particularly Examples 7-13, wherein the one or more UV light sources are optically coupled at any location with the transparent substrate and/or the light-emitting layer.

Example 15: The article of any examples herein, particularly Examples 1-14, wherein the light-emitting layer comprises one or more polymer layers.

Example 16: The article of any examples herein, particularly Example 15, wherein one of the one or more polymer layers is a sealing layer.

Example 17: The article of any examples herein, particularly Example 15 or 16, wherein one of the one or more of the polymer layers is configured to substantially prevent UV light absorption.

Example 18: The article of any examples herein, particularly Examples 1-17, wherein the article is a ship hull, lenses, windows, sensors, surgical table, optical equipment, heat exchanger, medical equipment, aerospace equipment, bathroom equipment, oceanographical equipment, appliances, wound healing article, or a combination thereof.

Example 19: The article of any examples herein, particularly Examples 1-18 wherein when the article is exposed to a biofouling environment such that at least a portion of the light-emitting layer faces the biofouling environment, the article exhibits reduced biofouling as compared to a substantially identical reference article in the absence of the light-emitting layer.

Example 20: The article of any examples herein, particularly Examples 1-19, wherein the article is disinfecting.

Example 21: The article of any one of Examples 1-20, wherein the article is anti-biofouling.

Example 22: A disinfectant system comprising the article of any examples herein, particularly Examples 1-21.

Example 23: A method of comprising: forming an article by coupling one or more UV light sources such that a light beam is side-emitted by a light-emitting layer in a direction substantially perpendicular to the direction of the light beam; wherein the light-emitting layer comprises a plurality of light-scattering elements and exhibits at least 90% transmittance; wherein the article is at least partially disinfecting and/or anti-biofouling.

Example 24: The method of any examples herein, particularly Example 23, wherein the light-emitting layer is disposed on a transparent or non-transparent substrate.

Example 25: The method of any examples herein, particularly Examples 23-24, wherein the light-emitting layer comprises one or more polymer layers.

Example 26: The method of any examples herein, particularly Example 25, wherein the plurality of light-scattering elements at least partially are embedded in one or more polymer layers.

Example 27: The method of any examples herein, particularly Examples 23-26, wherein the plurality of light-scattering elements are uniformly dispersed in the light-emitting layer.

Example 28: The method of any examples herein, particularly Examples 23-27, wherein the light-emitting layer comprises a concentration gradient of the plurality of light-scattering elements along a length, width, and/or depths of the light-emitting layer.

Example 29: The method of any examples herein, particularly Examples 23-28, wherein the light-emitting layer comprises a localized dispersion of the plurality of light-scattering elements.

Example 30: The method of any examples herein, particularly Examples 23-29, wherein the plurality of light-scattering elements comprise a plurality of nanoparticles.

Example 31: The method of any examples herein, particularly Examples 24-30, wherein the light-emitting layer is formed by embedding a plurality of nanoparticles, forming defects, air bubbles, or a combination thereof within at least a portion of a surface of the substrate, wherein the substrate is transparent.

Example 32: The method of any examples herein, particularly Examples 25-31, wherein one of the one or more polymer layers is a sealing layer.

Example 33: The method of any examples herein, particularly Examples 25-32, wherein at least one of the one or more of the polymer layers is configured to substantially prevent UV light absorption.

Example 34: The method of any examples herein, particularly Examples 23-33, wherein the plurality of light-scattering elements comprise a plurality of silica nanoparticles having a size of 50 nm to 500 nm.

Example 35: The method of any examples herein, particularly Examples 23-34, wherein the one or more UV light sources comprise an LED.

Example 36: The method of any examples herein, particularly Examples 23-35, wherein the one or more UV light sources operate at the same or different wavelengths.

Example 37: The method of any examples herein, particularly Examples 23-36, wherein the light-emitting layer has a thickness of 50 nm to 5 cm.

Example 38: The method of any examples herein, particularly Examples 24-37, wherein the one or more UV light sources are optically coupled at any location with the transparent substrate and/or the light-emitting layer.

Example 39: The method of any examples herein, particularly Examples 23-38, wherein the article is a ship hull, lenses, windows, sensors, surgical table, optical equipment, heat exchanger, medical equipment, aerospace equipment, bathroom equipment, oceanographical equipment, appliances, wound healing article, or a combination thereof.

Example 40: A method comprising: forming an article of any examples herein, particularly Examples 1-22; exposing the article to a biofouling environment such that the light-emitting layer at least partially faces the biofouling environment, passing a light beam from the one or more UV-light sources to initiate a side emission of the UV light, and reducing biofouling at at least a portion of a surface of the article that is exposed to the biofouling environment.

Example 41: A biofouling-resistant device, comprising: a transparent media having an outer surface, the outer surface having a light dispersive coating deposited thereon; and a UV light source optically connected to the transparent media; whereby the dispersive coating disperses UV light emitted from the UV light source onto the outer surface of the transparent media thereby reducing biofouling.

Example 42: The device of any examples herein, particularly Example 41, wherein the UV light source emits light in wavelengths between about 250 nm and about 280 nm.

Example 43: The device of any examples herein, particularly Example 41 or 42, wherein the UV light source emits light in wavelength at about 265 nm.

Example 44: The device of any examples herein, particularly Examples 41-43, wherein the light dispersive coating comprises silica nanoparticles.

Example 45: The device of any examples herein, particularly Example 44, wherein the silica nanoparticles comprise diameters having at least 50 nm.

Example 46: The device of any examples herein, particularly Example 45, wherein the silica nanoparticles comprise diameters having at least 100 nm.

Example 47: The device of any examples herein, particularly Example 46, wherein the silica nanoparticles comprise diameters having at least 200 nm.

Example 48: The device of any examples herein, particularly Example 47, wherein the silica nanoparticles have a concentration from about 2.65 μg/cm² to about 265 g/cm².

Example 49: The device of any examples herein, particularly Example 48, wherein the silica nanoparticles have a concentration of about 26.5 g/cm².

Example 50: The device of any examples herein, particularly Example 41-49, further comprising a silicone binder coating overlaying the light dispersive coating.

Example 51: The device of any examples herein, particularly Example 41-50, wherein the UV light source is an LED.

Example 52: A method of making a biofouling-resistant surface, comprising: providing a transparent media having an outer surface; coating the outer surface with a light dispersive coating; and optically coupling a UV light source of the transparent media; whereby the light dispersive coating disperses UV light emitted from the UV light source onto the outer surface of the transparent media thereby reducing biofouling.

Example 53: The method of any examples herein, particularly Example 52, wherein the light dispersive coating is electrostatically deposited onto the transparent media.

Example 54: The method of any examples herein, particularly Example 52 or 53, further comprising applying a silicone binder to the light dispersive coating.

Example 55: The method of any examples herein, particularly Example 52-54, wherein the UV light source is configured and arranged to emit light in wavelengths between about 250 nm and about 280 nm.

Example 56: The method of any examples herein, particularly Example 52-55, wherein the UV light source emits light in wavelength at about 265 nm.

Example 57: The method of any examples herein, particularly Example 52-56, wherein the light dispersive coating comprises silica nanoparticles from about 50 nm in diameter to about 200 nm in diameter.

Example 58: The method of any examples herein, particularly Example 57, wherein the silica nanoparticles have a concentration from about 2.65 μg/cm² to about 265 μg/cm².

Example 59: The method of any examples herein, particularly Example 58, wherein the silica nanoparticles have a concentration of about 26.5 μg/cm².

Example 60: The method of any examples herein, particularly Example 52-59, wherein the UV light source is an LED.

1. An article comprising
a) a light-emitting layer having a thickness of 50 nm to 5 cm; and
b) one or more UV light sources coupled within the article such that a light beam is side-emitted by the light-emitting layer in a direction substantially perpendicular to a direction of the light beam;
wherein the light-emitting layer comprises a plurality of light-scattering elements and exhibits at least 90% transmittance.

2. The article of claim 1, wherein the plurality of light-scattering elements comprise a plurality of nanoparticles comprising silica nanoparticles having a size of 50 nm to 500 nm.

3. (canceled)

4. (canceled)

5. The article of claim 1, wherein the light-emitting layer is positioned on a transparent or non-transparent substrate, wherein when the substrate is transparent, and the light-emitting layer is
i) disposed on a surface of the substrate, and/or
ii) at least partially embedded into a body of the transparent substrate adjacent to the surface of the substrate and wherein the plurality of light-scattering elements comprise one or more of a plurality of particles, defects, air bubbles, or a combination thereof,
wherein the substrate has a regular or irregular geometry; and wherein, and
wherein at least a portion of the light-emitting layer is optionally substantially conforms to at least one surface of the substrate.

6. (canceled)

7. The article of claim 5, wherein the one or more UV light sources are optically coupled with the transparent substrate and/or the light-emitting layer.

8. The article of claim 7 wherein the transparent substrate behaves as an optical waveguide such that the light beam propagates along a longitudinal axis of the transparent substrate, and wherein the transparent substrate has a thickness of 250 microns to 50 cm.

9. (canceled)

10. (canceled)

11. (canceled)

12. (canceled)

13. (canceled)

14. (canceled)

15. The article of claim 1, wherein the light-emitting layer comprises one or more polymer layers, wherein one of the one or more polymer layers is a sealing layer and/or wherein one of the one or more of the polymer layers is configured to substantially prevent UV light absorption.

16. (canceled)

17. (canceled)

18. The article of claim 1, wherein the article is a ship hull, lenses, windows, sensors, surgical table, optical equipment, heat exchanger, medical equipment, aerospace equipment, bathroom equipment, oceanographical equipment, appliances, wound healing article, or a combination thereof.

19. The article of claim 1, wherein when the article is exposed to a biofouling environment such that at least a portion of the light-emitting layer faces the biofouling environment, the article exhibits reduced biofouling as compared to a substantially identical reference article in the absence of the light-emitting layer.

20. (canceled)

21. (canceled)

22. A disinfectant system comprising the article of claim 1.

23. A method comprising:
forming an article by coupling one or more UV light sources such that a light beam is side-emitted by a light-emitting layer in a direction substantially perpendicular to the direction of the light beam; wherein the light-emitting layer has a thickness of 50 nm to 5 cm;
wherein the light-emitting layer comprises a plurality of light-scattering elements and exhibits at least 90% transmittance;
wherein the article is at least partially disinfecting and/or anti-biofouling.

24. The method of claim 23, wherein the light-emitting layer is disposed on a transparent or non-transparent substrate.

25. The method of claim 23, wherein the light-emitting layer comprises one or more polymer layers.

26. The method of claim 25, wherein the plurality of light-scattering elements at least partially are embedded in one or more polymer layers.

27. The method of claim 23, wherein
i) the plurality of light-scattering elements uniformly dispersed in the light-emitting layer; and/or
ii) wherein the light-emitting layer comprises a concentration gradient of the plurality of light-scattering elements along a length, width, and/or depths of the light-emitting layer; and/or
iii) wherein the light-emitting layer comprises a localized dispersion of the plurality of light-scattering elements.

28. (canceled)

29. (canceled)

30. The method of claim 23, wherein the plurality of light-scattering elements comprise a plurality of nanoparticles comprising a plurality of silica nanoparticles having a size of 50 nm to 500 nm.

31. The method of claim 24, wherein the light-emitting layer is formed by embedding a plurality of nanoparticles, forming defects, air bubbles, or a combination thereof within at least a portion of a surface of the substrate, wherein the substrate is transparent.

32. The method of claim 25, wherein one of the one or more polymer layers is a sealing layer; and/or wherein at least one of the one or more of the polymer layers is configured to substantially prevent UV light absorption.

33. (canceled)

34. (canceled)

35. (canceled)

36. (canceled)

37. (canceled)

38. The method of claim 24, wherein the one or more UV light sources are optically coupled at any location with the transparent substrate and/or the light-emitting layer.

39. The method of claim 23, wherein the article is a ship hull, lenses, windows, sensors, surgical table, optical equipment, heat exchanger, medical equipment, aerospace equipment, bathroom equipment, oceanographical equipment, appliances, wound healing article, or a combination thereof.

40. A method comprising:
forming an article of claim 1;
exposing the article to a biofouling environment such that the light-emitting layer at least partially faces the biofouling environment,
passing a light beam from the one or more UV-light sources to initiate a side emission of the UV light and
reducing biofouling at at least a portion of a surface of the article that is exposed to the biofouling environment.
